(19) United States
(12) Patent Application Publication
(10) Pub. No.: US 2023/0277564 A1
(43) Pub. Date: Sep. 7, 2023

(54) CANNABINOIDAL COMPOSITIONS FOR THE TREATMENT OF DISEASE

(71) Applicant: The State of Israel, Ministry of Agriculture & Rural Development, Agricultural Research Organization, Rishon-LeZion (IL)

(72) Inventors: Hinanit KOLTAI, Rishon-LeZion (IL); Nurit SHALEV, Tirat Yehuda (IL); Dvora NAMDAR, Beit Gamliel (IL); Guy MECHREZ, Adi (IL)

(73) Assignee: The State of Israel, Ministry of Agriculture & Rural Development, Agricultural Research Organization, Rishon-LeZion (IL)

(21) Appl. No.: 18/130,497

(22) Filed: Apr. 4, 2023

Related U.S. Application Data
(63) Continuation of application No. PCT/IL2021/051191, filed on Oct. 4, 2021.
(60) Provisional application No. 63/087,232, filed on Oct. 4, 2020.

(51) Int. Cl. A61K 31/00; A61K 36/185; A61K 31/05; A61K 31/365; A61K 31/045; A61K 31/015; A61P 29/00; A61P 11/00

(52) U.S. Cl. CPC A61K 31/658; A61K 36/185; A61K 31/05; A61K 31/365; A61K 31/045; A61K 31/015; A61P 29/00; A61P 11/00

(57) ABSTRACT

Cannabinoidal compositions for the treatment of disease are provided. Accordingly, there is provided a method of treating a disease that can benefit from activating macrophages comprising administering to the subject a therapeutically effective amount of a composition comprising a cannabis derived fraction comprising phytocannabinoids, wherein said phytocannabinoids comprise at least 80% CBD and at least one of CBG and THCV. Also provided is a method of treating a cytokine storm comprising administering to the subject a therapeutically effective amount of a synthetic composition comprising phytocannabinoids, wherein said phytocannabinoids comprise at least 80% CBD and at least one of CBG and THCV.

### RELATED APPLICATIONS

This application is a National Phase of PCT Patent Application No. PCT/IL2021/051191 having International filing date of Oct. 4, 2021, which claims the benefit of priority under 35 USC § 119(e) of U.S. Provisional Patent Application No. 63/087,232 filed on Oct. 4, 2020. The contents of the above applications are all incorporated by reference as if fully set forth herein in their entirety.

### SEQUENCE LISTING STATEMENT

The XML file, entitled 96193SequenceListing.xml, created on Apr. 4, 2023, comprising 23,917 bytes, submitted concurrently with the filing of this application is incorporated herein by reference.

### FIELD AND BACKGROUND OF THE INVENTION

The present invention, in some embodiments thereof, relates to cannabinoidal compositions for the treatment of disease.

The worldwide viral pandemic termed COVID-19, caused by the new Corona virus, SARS-CoV-2 and its variants, is associated with significant morbidity and mortality with collateral effects on culture and economics. Respiratory failure due to acute respiratory distress syndrome (ARDS) is the leading cause of mortality [Mehta, P. et al. Lancet (London, England) 395, 1033 (2020)].

COVID-19 disease progression is often characterized by a two-phase immune responses. A specific adaptive immune response is required at the first phase to eliminate the virus and to prevent disease progression to severe stages [Shi, Y., et al., Cell Death Differ. 27, 1451-1454 (2020)]. Therefore, strategies to increase immune responses at this first stage are critical. The second phase is usually associated with a virally induced cytokine storm syndrome [[Mehta, P. et al. Lancet (London, England) 395, 1033 (2020); Shi, Y., et al., Cell Death Differ. 27, 1451-1454 (2020)]. The cytokine storm syndrome is characterized by elevated levels of several cytokines including interleukin 6 (IL-6) and interleukin 8 (IL-8), tumor necrosis factor (TNF) and C-C Motif Chemokine Ligand 2 (CCL2) [Ruscitti, P. et al. Autoimmun. Rev. 19, 102562 (2020)]. Specific to the respiratory system, lung epithelial cells were suggested to play a crucial role in the release of several pro-inflammatory cytokines such as IL-6 and IL-8 [Gao, Y. et al. J. Med. Virol. 92, 791-796 (2020)].

Marijuana (Cannabis sativa) contains more than 500 constituents, among them phytocannabinoids, terpenes and flavonoids [ElSohly et al., Phytochemistry of Cannabis sativa L. Phytocannabinoids, Springer (2017) 1-36].

Cannabinoids were previously suggested as immune modulators and were shown to change the balance between pro- and anti-inflammatory cytokines and influence macrophage activity (see e.g. Oláh, A. et al. Front. Immunol. 8, 1487 (2017); Gonçalves, E. D. & Dutra, R. C. Drug Discov. Today 24, 1845-1853 (2019); Romano, B. et al. Pharmacol. Res. 113, 199-208 (2016); Friedman, M., et al. Proc. Soc. Exp. Biol. Med. 182, 225-228 (1986)].

Additional background art includes:

International Patent Application Publication No. WO2020/121312; WO2018/163164; and WO2018/163163.

### SUMMARY OF THE INVENTION

According to an aspect of some embodiments of the present invention there is provided a method of treating a disease that can benefit from activating macrophages, wherein the disease is not cancer, in a subject in need thereof, the method comprising administering to the subject a therapeutically effective amount of a composition comprising a cannabis derived fraction comprising phytocannabinoids, wherein the phytocannabinoids comprise at least 80% CBD and at least one of CBG and THCV, wherein when the phytocannabinoids comprise the CBG the phytocannabinoids comprise at least 2% CBG, thereby treating the disease in the subject.

According to an aspect of some embodiments of the present invention there is provided a composition comprising a cannabis derived fraction comprising phytocannabinoids, wherein the phytocannabinoids comprise at least 80% CBD and at least one of CBG and THCV, wherein when the phytocannabinoids comprise the CBG the phytocannabinoids comprise at least 2% CBG, for use in treating a disease that can benefit from activating macrophages, wherein the disease is not cancer, in a subject in need thereof.

According to some embodiments of the invention, the disease is selected from the group consisting of infectious disease, systemic lupus erythematosus, rheumatoid arthritis, multiple sclerosis, a lipid metabolism disorder, atherosclerosis, obstructive jaundice, cholestatic liver disease, skin xanthoma, xanthelasma, tuberous xanthoma, xanthoma striatum, vanishing bile duct syndrome.

According to some embodiments of the invention, the disease is an infectious disease.

According to some embodiments of the invention, the infectious disease is at a stage prior to cytokine storm.

According to some embodiments of the invention, the infectious disease is associated with a viral infection.

According to some embodiments of the invention, the cannabis derived fraction has a pro-inflammatory effect on macrophages.

According to some embodiments of the invention, the pro-inflammatory effect is manifested by at least one of:
(i) macrophage polarization;
(ii) macrophage phagocytosis;
(iii) expression and/or secretion of pro-inflammatory cytokines; and/or
(iv) expression of receptors that are associated with phagocytosis.

According to some embodiments of the invention, the cannabis derived fraction has an anti-inflammatory effect on lung epithelial cells.

According to an aspect of some embodiments of the present invention there is provided a method of treating a cytokine storm in a subject in need thereof, the method comprising administering to the subject a therapeutically effective amount of a synthetic composition comprising phytocannabinoids, wherein the phytocannabinoids comprise at least 80% CBD and at least one of CBG and THCV, thereby treating the disease in the subject.

According to an aspect of some embodiments of the present invention there is provided a synthetic composition comprising phytocannabinoids, wherein the phytocannabinoids comprise at least 80% CBD and at least one of CBG and THCV, for use in treating a cytokine storm in a subject in need thereof.

According to some embodiments of the invention, when the phytocannabinoids comprise the CBG the phytocannabinoids comprise at least 2% CBG.

According to some embodiments of the invention, the cytokine storm is associated with a viral infection.

According to some embodiments of the invention, the composition has an anti-inflammatory effect on lung epithelial cells.

According to some embodiments of the invention, the anti-inflammatory effect is manifested by at least one of:
(i) reduction of expression and/or secretion of pro-inflammatory cytokines; and/or
(ii) reduction of expression of a receptor associated with the dual renin-angiotensin system (RAS) and/or viral infection.

According to some embodiments of the invention, the pro-inflammatory cytokines are selected from the group consisting of IL-6, IL-8, IL-7, CCL2 and CCL7.

According to some embodiments of the invention, the composition has a combined additive or synergistic anti-inflammatory effect on lung epithelial cells as compared to each of the CBD, CBG and THCV when administered as a single agent.

According to some embodiments of the invention, the composition has a combined additive or synergistic pro-inflammatory effect on macrophages as compared to each of the CBD, CBG and THCV when administered as a single agent.

According to some embodiments of the invention, the viral infection is a respiratory viral infection.

According to some embodiments of the invention, the respiratory viral infection is selected from the group consisting of a Corona virus infection, a respiratory syncytial virus (RSV) infection, an influenza virus infection, a parainfluenza virus infection, an adenovirus infection and a rhinovirus infection.

According to some embodiments of the invention, the viral infection is a Corona virus infection.

According to some embodiments of the invention, the Corona virus is SARS-CoV-2, Middle East respiratory syndrome Coronavirus (MERS-CoV) or severe acute respiratory syndrome Coronavirus (SARS-CoV).

According to some embodiments of the invention, the Corona virus is SARS-CoV-2.

According to some embodiments of the invention, the at least 80% CBD is at least 90%.

According to some embodiments of the invention, the phytocannabinoids comprise 90-95% CBD.

According to some embodiments of the invention, when the phytocannabinoids comprise the CBG the phytocannabinoids comprise at least 5% CBG.

According to some embodiments of the invention, when the phytocannabinoids comprise the CBG the phytocannabinoids comprise 5-7% CBG.

According to some embodiments of the invention, when the phytocannabinoids comprise the CBG the phytocannabinoids comprise 5.5-6.5% CBG.

According to some embodiments of the invention, when the phytocannabinoids comprise the CBG, a concentration ratio of the CBD and the CBG is 20: 1-10:1.

According to some embodiments of the invention, when the phytocannabinoids comprise the CBG, a concentration ratio of the CBD and the CBG is 17: 1-13:1.

According to some embodiments of the invention, when the phytocannabinoids comprise the THCV the phytocannabinoids comprise at least 0.2% THCV.

According to some embodiments of the invention, when the phytocannabinoids comprise the THCV the phytocannabinoids comprise less than 1% THCV.

According to some embodiments of the invention, when the phytocannabinoids comprise the THCV the phytocannabinoids comprise 0.3-0.5% THCV.

According to some embodiments of the invention, when the phytocannabinoids comprise the THCV, a concentration ratio of the CBD and the THCV is 300: 1-100:1.

According to some embodiments of the invention, when the phytocannabinoids comprise the THCV, a concentration ratio of the CBD and the THCV is 250: 1-200:1.

According to some embodiments of the invention, the composition is devoid of Tetrahydrocannabinol (THC).

According to some embodiments of the invention, the composition is devoid of phytocannabinoids other than the CBD, CBG and/or THCV.

According to some embodiments of the invention, the composition is devoid of cannabis active ingredients other than the phytocannabinoids.

According to some embodiments of the invention, the cannabis derived fraction comprises cannabis active ingredients other than the phytocannabinoids.

According to some embodiments of the invention, the cannabis derived fraction comprises at least one of the terpenes and terpenoids listed in Table 1.

According to some embodiments of the invention, the cannabis derived fraction comprises at least one of the terpenes and terpenoids listed in Table 1 in a concentration ratio according to Table 1±10%.

According to some embodiments of the invention, the cannabis derived fraction is a liquid chromatography fraction of a cannabis extract.

According to some embodiments of the invention, the liquid chromatography fraction is obtainable by subjecting the cannabis extract to flash chromatography comprising a Flash chromatography apparatus equipped with a diode array detector, a C18 functionalized silica column, a 85% methanol in water mobile phase, at a flow rate of 60 ml/min.

According to some embodiments of the invention, the fraction is collected between about 26-31 minutes of the flash chromatography.

According to some embodiments of the invention, the phytocannabinoids are synthetic.

According to some embodiments of the invention, the phytocannabinoids are purified from cannabis.

According to some embodiments of the invention, the cannabis is a cannabis strain Arbel.

According to some embodiments of the invention, presence or absence of the phytocannabinoids in the composition is effected by high pressure liquid chromatography (HPLC).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B demonstrate the effect of C. sativa Arbel crude extract and the indicated chromatography fractionated fractions on the levels of IL-6 (FIG. 1A) and IL-8 (FIG. 1B) secreted from A549 cells following treatment with 300 ng/mL TNFα. C. sativa extract and fractions were administered at a concentration of 5 μg/mL for 4 hours, following IL-6 and IL-8 levels were measured in the supernatant. Values (pg/cell) were normalized to cell number as determined in Alamar Blue fluorescence (resazurin) assay relatively to a TNFα-treated control. Dexamethasone (Dex; 4 μg/mL) served as a positive control; Control (methanol) treatment served as solvent (vehicle) control; TNFα (TNFα in methanol) treatment served as treatment control. Error bars indicate±s.e.m. (n=3). Levels with different letters are significantly different from all combinations of pairs by Tukey-Kramer honest significant difference (HSD; P≤0.05).

FIGS. 1C-D show dose-effect curves of C. sativa fraction F_{CBD} on IL-6 (FIG. 1C) and IL-8 (FIG. 1D) levels secreted from A549 cells. Data points were connected by non-linear regression lines of the sigmoidal dose-response relation. GraphPad Prism was used to produce the dose-response curve and IC₅₀ doses. Error bars indicate ±s.e.m. (n=3).

FIGS. 1E-F show dose-effect curves of synthetic composition mimicking fraction F_{CBD} (hereinafter F_{CBD:std} which comprises 93.5% CBD+6.1% CBG+0.4% THCV) on IL-6 (FIG. 1E) and IL-8 (FIG. 1F) levels secreted from A549 cells. Data points were connected by non-linear regression lines of the sigmoidal dose-response relation. GraphPad Prism was used to produce the dose-response curve and IC₅₀ doses. Error bars indicate ±s.e.m. (n=3).

FIGS. 2A-B demonstrate IL-6 (FIG. 2A) and IL-8 (FIG. 2B) levels secreted from A549 cells treated with F_{CBD} or CBD. Cells were treated with 300 ng/mL TNFα, 4.1 μg/mL F_{CBD} (F_{CBD}) or CBD at the indicated concentrations. IL-8 and IL-6 levels were measured in the cell supernatant 4 hours following treatment. Values (pg/cell) were normalized to cell number as determined in Alamar Blue fluorescence (resazurin) assay relatively to a TNFα-treated control. Dexamethasone (Dex; 4 μg/mL) served as a positive control; Control (methanol) treatment served as solvent (vehicle) control; TNFα (TNFα in methanol) served as treatment control. Error bars indicate ±s.e.m. (n=3). Levels with different letters are significantly different from all combinations of pairs by Tukey-Kramer honest significant difference (HSD; P<0.05).

FIG. 2C—demonstrates IL-8 levels secreted from A549 cells treated with F_{CBD:std} at the indicated concentrations or CBD, CBG, THCV, a mix of CBD+CBG or a mix of CBD+THCV at concentration equivalent to the concentration in 10 μg/mL F_{CBD}:std. Cells were treated with 300 ng/mL TNFα, and the indicated phytocannabinoid composition. IL-8 and IL-6 levels were measured in the cell supernatant 4 hours following treatment. Values (pg/cell) were normalized to cell number as determined in Alamar Blue fluorescence (resazurin) assay relatively to a TNFα-treated control. Dexamethasone (Dex; 4 μg/mL) served as a positive control; Methanol served as solvent (vehicle) control; TNFα in methanol served as TNFα control. Error bars indicate ±s.e.m. (n=3). Levels with different letters are significantly different from all combinations of pairs by Tukey-Kramer honest significant difference (HSD; P<0.05).

FIGS. 3A-B demonstrate IL-6 (FIG. 3A) and IL-8 (FIG. 3B) levels secreted from A549 cells treated with F_{CBD} or F_{CBD}-std with or without CB1 or CB2 inverse agonists (IA) or TRPA1 blocker. Cells were treated with 300 ng/mL TNFα and F_{CBD} (F_{CBD}) or F_{CBD}-std (F_{CBD}-std) at a concentration of 3.4 and 4.1 μg/mL, respectively, in the presence or absence of IA of CB1 or CB2 or a TRPA1 blocker. IL-6 and IL-8 levels were measured in the cells supernatant 4 hours following treatment. Values (pg/cell) were normalized to cell number as determined via Alamar Blue fluorescence (resazurin) assay relatively to a TNFα-treated control. Dexamethasone (Dex) served as a positive control at 4 μg/mL; Control (methanol) treatment served as solvent (vehicle) control; TNFα (TNFα in methanol) served as treatment control. Error bars indicate ±s.e.m. (n=3). Levels with different letters are significantly different from all combinations of pairs by Tukey-Kramer honest significant difference (HSD; P<0.05).

FIGS. 4A-D demonstrate the results of quantitative PCR-based determination of RNA steady state levels of the CCL2 (FIG. 4A) CCL7, (FIG. 4B) IL-7 (FIG. 4C) or ACE2 (FIG. 4D) genes in A549 cells, 6 hours following treatment with TNFα (300 μg/mL) and F_{CBD} (F_{CBD}) at 7 μg/mL, or Dexamethasone (Dex) 4 μg/mL, relative to control. Gene transcript values were determined by quantitative PCR as a ratio between the target gene versus a reference gene (HPRT1; geneID 3251). Values were calculated relative to the average expression of target genes in treated versus control using the 2^{ΔΔct} method. Error bars indicate ±s.e.m. (n=3). Levels with different letters are significantly different from all combinations of pairs by Tukey-Kramer honest significant difference (HSD; P<0.05).

FIGS. 5A-C demonstrate the results of quantitative PCR-based determination of RNA steady state levels of the IL-6 (FIG. 5A) IL-8, (FIG. 5B) or CCL2 (FIG. 5C) genes in PMA-treated KG1 cells 6 hours following treatment with F_{CBD} (F_{CBD}) at 7 μg/mL, F_{CBD} std (F_{CBD}:std) at 7 μg/mL or Dexamethasone (Dex) at 8 μg/mL, relative to control. Gene transcript values were determined by quantitative PCR as a ratio between the target gene versus a reference gene (HPRT1; geneID 3251). Values were calculated relative to the average expression of target genes in treated versus control using the 2^{ΔΔct} method.

FIGS. 5D-E demonstrate the level of IL-8 secreted from KG1 cells treated with F_{CBD} or F_{CBD}-std. Cells were treated with 300 ng/mL TNFα and F_{CBD} (F_{CBD}) or F_{CBD} std (F_{CBD}:std) at 10 μg/mL (FIG. 5D) or at the indicated concentrations (FIG. 5E). IL-8 level was measured in the cells supernatant 4 hours following treatment. Values (pg/cell) were normalized to cell number as determined in Alamar Blue fluorescence (resazurin) assay relatively to a TNFα-treated control. Dexamethasone (Dex; 4 μg/mL) served as a positive control; Control (methanol) treatment served as solvent (vehicle) control; TNFα (TNFα in methanol) served as treatment control. Error bars indicate ±s.e.m. (n=3). Levels with different letters are significantly different from all combinations of pairs by Tukey-Kramer honest significant difference (HSD; P≤0.05).

FIG. 6 shows representative examples of confocal images of macrophages following treatment with F_{CBD} (7 μg/mL) as compared to solvent (vehicle) control. PMA-treated KG1 cells were treated with F_{CBD} or solvent control for 16 hours and then incubated for 4 hours with silica beads (SNP; 40 μg/mL). Following, cells were stained for F-actin (EasyProbes™ ActinRed 555 Stain, red stain, AP-FP032), and nuclei (Hoechst, blue stain, AP-FP027); n≥5, in each biological replicate multiple cells were examined (see Table 2 hereinbelow). Membrane filopodia-like structures are marked with white arrows.

FIGS. 7A-C demonstrate the results of quantitative PCR-based determination of RNA steady state levels of the FcγRII (FIG. 7A) CD36 (FIG. 7B) and SCARB1 (FIG. 7C) genes in PMA-treated KG1 cells following treatment with F_{CBD} at 7 μg/mL, F_{CBD:std} at 7 μg/mL, Ruxolitinib (Ruxo) at 100 μg/mL or Palmitic acid (PA) at 150 Gene transcript values were determined by quantitative PCR as a ratio between the target gene versus a reference gene (HPRT1; geneID 3251). Values were calculated relative to the average expression of target genes in treated versus control using the 2^{ΔΔct} method. Error bars indicate ±s.e.m. (n=3). Levels with different letters are significantly different from all combinations of pairs by Tukey-Kramer's honest significant difference (HSD; P≤0.05). *indicates significantly different mean from the control based on Student T-test (P≤0.05).

FIG. 8 shows the HPLC profile of C. sativa Arbel chromatography fractionated fractions F_{CBD} and F_{THC}. Composition of the fractions is given as percentage of total phytocannabinoid content in pie charts.

FIG. 9 demonstrates viability of A549 cells treated with crude C. sativa Arbel extract and the indicated fractions. Cells were treated with 300 ng/mL TNFα and C. sativa extract and fractions at a concentration of 5/mL for 4 hours. Cell number was determined using Alamar Blue fluorescence (resazurin assay). Dexamethasone (Dex; 4 μg/mL) served as a positive control; Control (methanol) treatment served as solvent (vehicle) control; TNFα (TNFα in methanol) served as treatment control. Error bars indicate ±s.e.m. (n=3). Levels with different letters are significantly different from all combinations of pairs by Tukey-Kramer honest significant difference (HSD; P≤0.05).

FIG. 10 shows gas chromatogram of fraction F_{CBD}, with the major peak annotated with compounds i.d.

FIGS. 11A-B show examples of Imaging Flow Cytometry plots of macrophages following treatment with F_{CBD} at 7 μg/mL, F_{CBD:std} at 7 μg/mL, CBD at 4.35 μg/mL and solvent (vehicle) control. Differentiated KG1 cells were treated for 16 hours with the indicated treatment and then incubated for 4 hours with silica beads (SNP, ENP or ENPG; 40 μg/mL). At least 4,000 cells for each treatment were analyzed using Amnis IDEAS software and the distribution of the cell internalization scores were plotted (marked in white lines under the curves). Cells with internalized SNP (FIG. 11A) and positive (pos) for ENP/ENPG (FIG. 11B) were gated as having internalization score higher than 0.33 or positive for silica beads, respectively.

FIGS. 11C-D show representative images of cells with internalized SNP beads (FIG. 11C) or on their surface (FIG. 11D), captured by the Amnis ImageStreamX. First column shows brightfield (BF) images of the cells, second column shows the SNP beads and third column shows the merged image (BF/SNP). Scale bar, 7 μm.

### DESCRIPTION OF SPECIFIC EMBODIMENTS OF THE INVENTION

The present invention, in some embodiments thereof, relates to cannabinoidal compositions for the treatment of disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Marijuana (Cannabis sativa) contains more than 500 constituents, among them phytocannabinoids, terpenes and flavonoids. Different constituents and preparations of marijuana have been suggested as immune modulators and were shown to change the balance between pro- and anti-inflammatory cytokines and influence macrophage activity.

The present inventor has now uncovered specific liquid chromatography fractions of cannabis inflorescence extracts and synthetic compositions mimicking same having anti-inflammatory activity on lung epithelial cells and differential effects on macrophage activity.

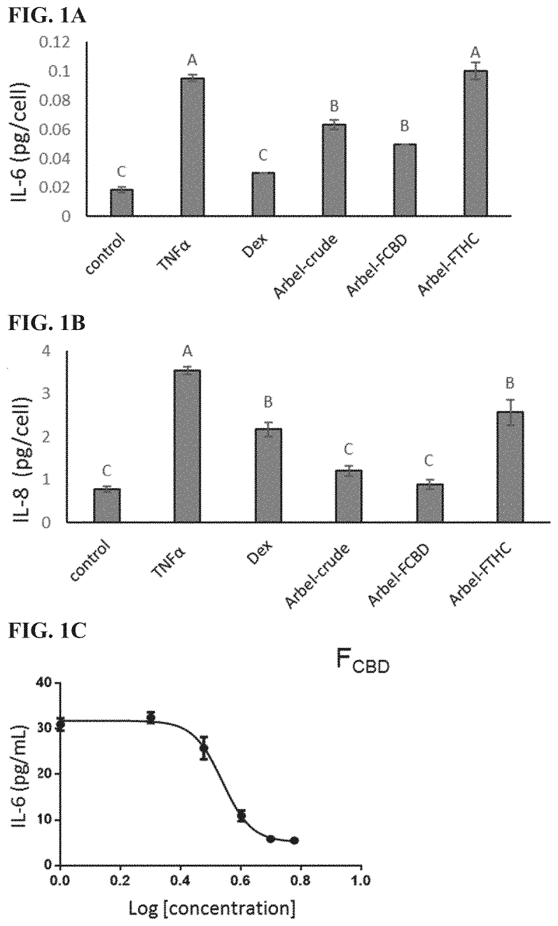

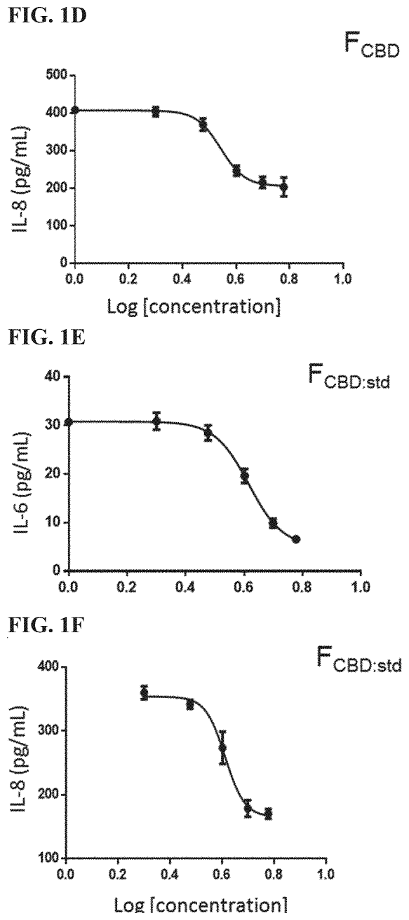

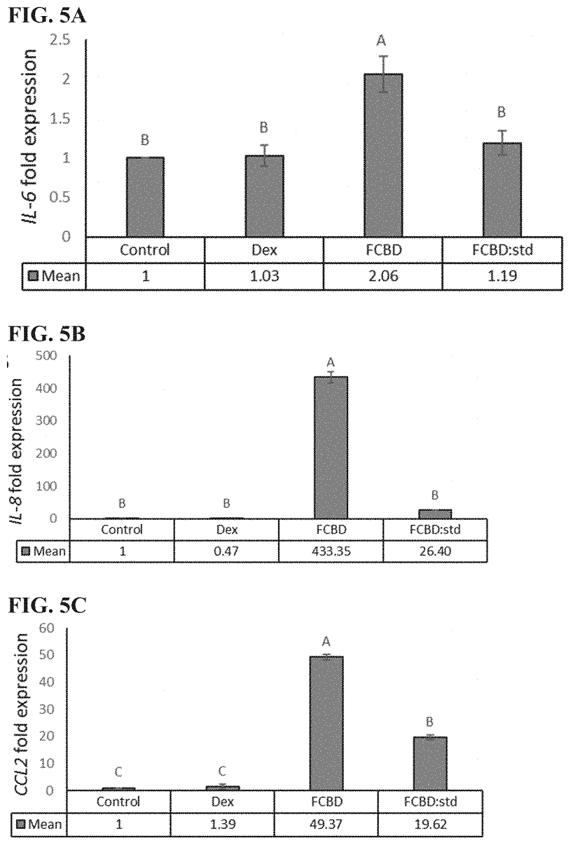

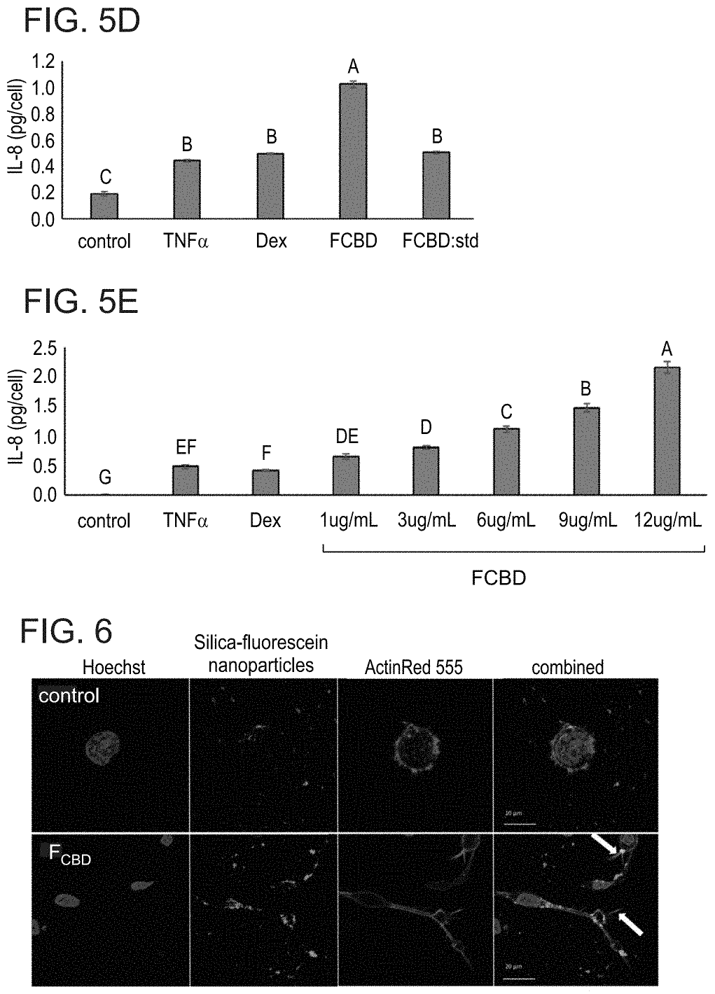

Specifically, as is illustrated in the Examples section which follows, the present inventors obtained inflorescences of C. sativa strain Arbel and fractionated them into several distinct fractions; among them a fraction referred to herein as high CBD (F_{CBD}) which significantly reduced inflammation induced by TNFα in lung epithelial cells, manifested by reduced secretion of IL-6 and IL-8, reduced expression of the pro-inflammatory cytokines CCL2 and CCL7, IL-7, and reduced expression of ACE2 (Example 1 of the Examples section which follows, FIGS. 1A-D, 4A-D and 8-10). A synthetic composition containing combination of phytocannabinoid standards at the ratios found in fraction F_{CBD} (referred to herein as F_{CBD:std}) had similar anti-inflammatory effects (Example 1 of the Examples section which follows, FIGS. 1E-F). This anti-inflammatory activity was superior and less cytotoxic compared to treatment with dexamethasone, the crude extract, other fractionated fractions and the phytocannabinoids comprised in the F_{CBD} fraction when administered as single compounds (Example 1 of the Example section which follows, FIGS. 1A-B, 2A-C, 8-9). Further, F_{CBD} induced macrophage activation manifested by secretion of pro-inflammatory cytokines, polarization and phagocytosis; however, the synthetic F_{CBD:std} composition had a significantly reduced effect on macrophage activation (Example 2 of the Examples section which follows, FIGS. 5A-E, 6, 7A-C and 11A-D).

Consequently, specific embodiments of the present invention propose novel cannabis derived fractions for use in treating a disease that can benefit from activating macrophages (e.g. Corona virus infection prior to cytokine storm); while other specific embodiments of the present invention propose novel synthetic compositions comprising phytocannabinoids for use in treating cytokine storm (e.g. following Corona virus infection).

Thus, according to an aspect of the present invention, there is provided a method of treating a disease that can benefit from activating macrophages, wherein the disease is not cancer, in a subject in need thereof, the method comprising administering to the subject a therapeutically effective amount of a composition comprising a cannabis derived fraction comprising phytocannabinoids, wherein said phytocannabinoids comprise at least 80% CBD and at least one of CBG and THCV, wherein when said phytocannabinoids comprise said CBG said phytocannabinoids comprise at least 2% CBG, thereby treating the disease in the subject.

According to an additional or an alternative aspect of the present invention, there is provided a composition comprising a cannabis derived fraction comprising phytocannabinoids, wherein said phytocannabinoids comprise at least 80% CBD and at least one of CBG and THCV, wherein when said phytocannabinoids comprise said CBG said phytocannabinoids comprise at least 2% CBG, for use in treating a disease that can benefit from activating macrophages, wherein the disease is not cancer, in a subject in need thereof.

According to an additional or an alternative aspect of the present invention, there is provided a method of treating a cytokine storm in a subject in need thereof, the method comprising administering to the subject a therapeutically effective amount of a synthetic composition comprising phytocannabinoids, wherein said phytocannabinoids comprise at least 80% CBD and at least one of CBG and THCV, thereby treating the disease in the subject.

According to an additional or an alternative aspect of the present invention, there is provided a synthetic composition comprising phytocannabinoids, wherein said phytocannabinoids comprise at least 80% CBD and at least one of CBG and THCV, for use in treating a cytokine storm in a subject in need thereof.

According to a specific embodiment, the term “phytocannabinoid” refers to a meroterpenoid with a resorcinyl core typically decorated with a para-positioned isoprenyl, alkyl, or aralkyl side chain originated from a cannabis plant, acidic or decarboxylated acid forms thereof. The term also reads on synthetic analogs or derivatives of the plant originated substance.

Alternatively or additionally, the term “phytocannabinoid” refers to a cannabinoid selected from the list provided in Table 4 hereinbelow originated from a cannabis plant, acidic or decarboxylated acid forms thereof. The term also reads on synthetic analogs or derivatives of the plant originated substance.

TABLE 4
List of phytocannabinoids (modified from Berman P, Futoran
K, Lewitus G M, Mukha D, Benami M, Shlomi T, Meiri D.
A new ESI-LC/MS approach for comprehensive metabolic
profiling of phytocannabinoids in Cannabis. Scientific
reports. 2018 Sep. 24; 8(1): 1-5, the contents of which
are fully incorporated herein by reference)

Cannabiorcol-C1 (CBNO)
CBND-C1 (CBNDO)*
(−)-Δ 9 - trans-Tetrahydrocannabiorcol-C1 (Δ9 -THCO)
Cannabidiorcol-C1 (CBDO)
Cannabiorchromene-C1 (CBCO)
(−)-Δ 8-trans-(6aR,10aR)-Tetrahydrocannabiorcol-C1 (Δ8-THCO)
Cannabiorcyclol C1 (CBLO)
CBG-C1 (CBGO)
Cannabinol (CBN)
CBND-C2
Delta-9-tetrahydrocannabinol Δ9-THC (THC)
Cannabidiol (CBD)
Δ8-THC-C2
CBL-C2
Bisnor-cannabielsoin-C1 (CBEO)
Cannabigerol (CBG)
Cannabivarin-C3 (CBNV)
Cannabinodivarin-C3 (CBNDV)
Δ9-trans-Tetrahydrocannabivarin Δ9-THCV (THCV)
(−)-Cannabidivarin-C3 (CBDV)
(±)-Cannabichromevarin-C3 (CBCV)
(−)-Δ8-trans-THC-C3 (Δ8-THCV)
Δ7-tetrahydrocannabivarin-C3 (Δ7-THCV)
(±)-(1aS,3aR,8bR,8cR)-Cannabicyclovarin-C3 (CBLV)
2-Methyl-2-(4-methyl-2-pentenyl)-7-propyl-2H-1-benzopyran-5-ol
CBE-C2
Cannabigerovarin-C3 (CBGV)
Cannabitriol-C1 (CBTO)
Cannabinol-C4 (CBN-C4)
CBND-C4
(−)-Δ9-trans-Tetrahydrocannabinol-C4 (Δ9-THC-C4)
Cannabidiol-C4 (CBD-C4)
Cannabichromene (CBC)
(−)-trans-Δ8-THC-C4
CBL-C4
Cannabielsoin-C3 (CBEV)
CBG-C4
CBT-C2
Cannabichromanone-C3
Cannabiglendol-C3 (OH-iso-HHCV-C3)
Cannabioxepane-C5 (CBX)
Dehydrocannabifuran-C5 (DCBF)
Cannabinol-C5 (CBN)
Cannabinodiol-C5 (CBND)
Cannabifuran-C5 (CBF)
(−)-Δ9-trans-Tetrahydrocannabinol-C5 (Δ9-THC)
(−)-Δ8-trans-(6aR,10aR)-Tetrahydrocannabinol-C5 (Δ8-THC)
(±)-Cannabichromene-C5 (CBC)
(−)-Cannabidiol-C5 (CBD)
(±)-(1aS,3aR,8bR,8cR)-Cannabicyclol-C5 (CBL)
Cannabicitran-C5 (CBR)
(−)-Δ9-(6aS,10aR-cis)-Tetrahydrocannabinol-C5 ((−)-cis-Δ9-THC)
(−)-Δ7-trans-(1R,3R,6R)-Isotetrahydrocannabinol-C5 (trans-iso-Δ7-THC)
CBE-C4
Cannabigerol-C5 (CBG)
Cannabitriol-C3 (CBTV)
Cannabinol methyl ether-C5 (CBNM)
CBNDM-C5
8-OH-CBN-C5 (OH-CBN)
OH-CBND-C5 (OH-CBND)
10-Oxo-Δ6a(10a)-Tetrahydrocannabinol-C5 (OTHC)
Cannabichromanone D-C5
Cannabicoumaronone-C5 (CBCON-C5)
Cannabidiol monomethyl ether-C5 (CBDM)
Δ9-THCM-C5
(±)-3″-hydroxy-Δ4″-cannabichromene-C5
(5aS,6S,9R,9aR)-Cannabielsoin-C5 (CBE)
2-geranyl-5-hydroxy-3-n-pentyl-1,4-benzoquinone-C5
8α-Hydroxy-Δ9-Tetrahydrocannabinol-C5 (8α-OH-Δ9-THC)
8β-Hydroxy-Δ9-Tetrahydrocannabinol-C5 (8β-OH-Δ9-THC)
10α-Hydroxy-Δ8-Tetrahydrocannabinol-C5 (10α-OH-Δ8-THC)
10β-Hydroxy-Δ8-Tetrahydrocannabinol-C5 (10β-OH-Δ8-THC)
10α-hydroxy-Δ9,11-hexahydrocannabinol-C5
9β,10β-Epoxyhexahydrocannabinol-C5
OH-CBD-C5 (OH-CBD)
Cannabigerol monomethyl ether-C5 (CBGM)
Cannabichromanone-C5
CBT-C4
(±)-6,7-cis-epoxycannabigerol-C5
(±)-6,7-trans-epoxycannabigerol-C5
(−)-7-hydroxycannabichromane-C5
Cannabimovone-C5
(−)-trans-Cannabitriol-C5 ((−)-trans-CBT)
(+)-trans-Cannabitriol-C5 ((+)-trans-CBT)
(±)-cis-Cannabitriol-C5 ((±)-cis-CBT)
(−)-trans-10-Ethoxy-9-hydroxy-Δ6a(10a)-tetrahydrocannabivarin-C3 [(−)-
trans-CBT-OEt]
(−)-(6aR,9S,10S,10aR)-9,10-Dihydroxyhexahydrocannabinol-C5 [(−)-
Cannabiripsol] (CBR)
Cannabichromanone C-C5
(−)-6a,7,10a-Trihydroxy-Δ9-tetrahydrocannabinol-C5 [(−)-Cannabitetrol]
(CBTT)
Cannabichromanone B-C5
8,9-Dihydroxy-Δ6a(10a)-tetrahydrocannabinol-C5 (8,9-Di-OHCBT)
(±)-4-acetoxycannabichromene-C5
2-acetoxy-6-geranyl-3-n-pentyl-1,4-benzoquinone-C5
11-Acetoxy-Δ9-Tetrahydrocannabinol-C5 (11-OAc-Δ9-THC)
5-acetyl-4-hydroxycannabigerol-C5
4-acetoxy-2-geranyl-5-hydroxy-3-npentylphenol-C5
(−)-trans-10-Ethoxy-9-hydroxy-Δ6a(10a)-tetrahydrocannabinol-C5 ((−)-
trans-CBTOet)
4-acetoxy-2-geranyl-5-hydroxy-3-npropylphenol-C5
sesquicannabigerol-C5 (SesquiCBG)
carmagerol-C5
4-terpenyl cannabinolate-C5
β-fenchyl-Δ9-tetrahydrocannabinolate-C5
α-fenchyl-Δ9-tetrahydrocannabinolate-C5
epi-bornyl-Δ9-tetrahydrocannabinolate-C5
bornyl-Δ9-tetrahydrocannabinolate-C5
α-terpenyl-Δ9-tetrahydrocannabinolate-C5
4-terpenyl-Δ9-tetrahydrocannabinolate-C5

According to specific embodiments, at least 50% of the cannabinoids or cannabis-derived compounds in the composition or fraction are phytocannabinoids.

According to specific embodiments, at least 55%, at least 60% or at least 65% of the cannabinoids or cannabis-derived compounds in the composition or fraction are phytocannabinoids.

According to specific embodiments, 50-100%, 60-90% or 60-80% of the cannabinoids or cannabis-derived compounds in the composition or fraction are phytocannabinoids.

According to specific embodiments, 60-70% of the cannabinoids or cannabis-derived compounds in the composition or fraction are phytocannabinoids.

The compositions and fractions disclosed herein comprise phytocannabinoids wherein the phytocannabinoids comprise CBD and at least one of CBG and THCV.

According to specific embodiments, the phytocannabinoids in the composition or fraction comprise CBD and CBG.

According to specific embodiments, the phytocannabinoids in the composition or fraction comprise CBD and THCV.

According to specific embodiments, the phytocannabinoids in the composition or fraction comprise CBD, CBG and THCV.

Cannabidiol (CBD) (CAS No. 13956-29-1), as used herein, encompasses native CBD (i.e. originating from the Cannabis plant), or synthetic or naturally occurring analogs or derivatives thereof. According to specific embodiments, any CBD analog may be used in accordance with specific embodiments of the present teachings as long as it comprises the anti-inflammatory and/or macrophage activating activities described herein (alone, or as part of a composition discussed herein).

Exemplary CBD analogs include, but are not limited to, (−)-DMH-CBD-11-oic acid, HU-308 (commercially available e.g. from Tocris Bioscience, 3088), O-1602 (commercially available e.g. from Tocris Bioscience 2797/10), DMH-CBD (commercially available e.g. from Tocris Bioscience, 1481) [as discussed in detail in Burstein S, Bioorg Med Chem. (2015) 23(7): 1377-85], Abn-CBD, HUF-101. CBDV, CBDM, CBND-05, CBND-C3, 6-Hydroxy-CBD-triacetate or CBD-aldehyde-diacetate [as discussed in detail in An Overview on Medicinal Chemistry of Synthetic and Natural Derivatives of Cannabidiol, Frontiers in Pharmacology, June 2017|Volume 8|Article 422].

According to specific embodiments, the CBD is not CBDV.

According to specific embodiments, the CBD comprises native CBD.

Pure or synthetic CBD can be commercially obtained from e.g. Restek catalog no. 34011.

Cannabigerol (CBG) (CAS No. 25654-31-3), as used herein, encompasses native CBG (i.e. originating from the Cannabis plant), or synthetic or naturally occurring analogs or derivatives thereof. According to specific embodiments, any CBG analog may be used in accordance with specific embodiments of the present teachings as long as it comprises the anti-inflammatory and/or macrophage activating activities described herein (alone, or as part of a composition discussed herein).

According to specific embodiments, the CBG comprises native CBG.

Pure or synthetic CBG can be commercially obtained from e.g. Restek catalog no. 34091.

Tetrahydrocannabivarin (THCV) (CAS No. 31262-37-0), as used herein, encompasses native THCV (i.e. originating from the Cannabis plant), or synthetic or naturally occurring analogs or derivatives thereof. According to specific embodiments, any THCV analog may be used in accordance with specific embodiments of the present teachings as long as it comprises the anti-inflammatory and/or macrophage activating activities described herein (alone, or as part of a composition discussed herein).

An exemplary THCV analog include, but is not limited to, Δ8-THCV.

According to specific embodiments, the THCV comprises native THCV.

Pure or synthetic THCV can be commercially obtained from e.g. Restek catalog no. 34100.

As used herein, a “percent (%) of a phytocannabinoid” in the compositions or fractions disclosed herein refers to the concentration as presented in percentage (w/v) of the recited phytocannabinoid out of the total phytocannabinoids (and only the phytocannabinoids) in the composition or fraction, as can be determined by the peak area according to a HPLC profile of the composition.

Methods of determining presence or absence of a compounds in the composition or fraction, as well as the concentration of a compound in the composition are well known in the art, such as, but not limited to ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

According to specific embodiments, determining presence or absence of a compound in the composition or fraction and/or the concentration of a compound in the composition or fraction is effected by analytical high pressure liquid chromatography (HPLC).

According to specific embodiments, the phytocannabinoids comprise at least 80% CBD.

According to specific embodiments, the phytocannabinoids comprise at least 85%, at least 86%, at least 87%, at least 88%, at least 89%, at least 90%, at least 91%, at least 92% or at least 93% CBD.

According to specific embodiments, the phytocannabinoids comprise at least 90% CBD.

According to specific embodiments, the phytocannabinoids comprise less than 99%, less than 98%, less than 97%, less than 96% less than 95%, less than 94% CBD.

According to specific embodiments, the phytocannabinoids comprise less than 95% CBD.

According to specific embodiments, the phytocannabinoids comprise 93.5±10% CBD.

According to specific embodiments, the phytocannabinoids comprise 90-95% CBD.

According to specific embodiments, the phytocannabinoids comprise 93.5% CBD.

According to specific embodiments, the phytocannabinoids comprise at least 2% CBG.

According to specific embodiments, the phytocannabinoids comprise at least 3% CBG.

According to specific embodiments, the phytocannabinoids comprise at least 4%, at least 4.5%, at least 5%, at least 5.5% or at least 6% CBG.

According to specific embodiments, the phytocannabinoids comprise at least 5% CBG.

According to specific embodiments, the phytocannabinoids comprise less than 10%, less than 8%, less than 7% CBG.

According to specific embodiments, the phytocannabinoids comprise 2-10%, 3-9% or 4-8% CBG.

According to specific embodiments, the phytocannabinoids comprise 5-7% CBG.

According to specific embodiments, the phytocannabinoids comprise 5.5-6.5% CBG.

According to specific embodiments, the phytocannabinoids comprise about 6.1% CBG.

According to specific embodiments, the phytocannabinoids comprise at least 0.1% THCV.

According to specific embodiments, the phytocannabinoids comprise at least 0.2%, at least 0.25%, at least 0.3%, at least 0.35% THCV.

According to specific embodiments, the phytocannabinoids comprise less than 5%, less than 4%, less than 3%, less than 2%, less than 1%, less than 0.5% THCV.

According to specific embodiments, the phytocannabinoids comprise less than 1% THCV.

According to specific embodiments, the phytocannabinoids comprise 0.1-1%, 0.2-0.9%, 0.2-0.8%, 0.2-0.7% or 0.3-0.6% THCV.

According to specific embodiments, the phytocannabinoids comprise 0.3-0.5% THCV.

According to specific embodiments, the phytocannabinoids comprise about 0.4% THCV.

According to specific embodiments, a CBD:CBG concentration ratio in the composition or fraction is 20: 1-10:1.

According to specific embodiments, a CBD:CBG concentration ratio in the composition or fraction is 17: 1-13:1.

According to specific embodiments, a CBD:CBG concentration ratio in the composition or fraction is about 15:1.

According to specific embodiments, a CBD:THCV concentration ratio in the composition or fraction is 300: 1-100: 1.

According to specific embodiments, a CBD:THCV concentration ratio in the composition or fraction is 250: 1-200:1.

According to specific embodiments, a CBD:THCV concentration ratio in the composition or fraction is about 233:1.

According to specific embodiments, the concentration ratio is determined by g/l:g/l or μg/ml: μg/ml.

According to specific embodiments, the phytocannabinoids comprise 90-95% CBD, 5.5-6.5% CBG and 0.3-0.5% THCV.

According to a specific embodiment, the phytocannabinoids comprise about 93.5% CBD, about 6.1% CBG and about 0.4% THCV.

According to specific embodiments, the composition or fraction is devoid of THC.

According to specific embodiments, the composition or fraction is devoid of phytocannabinoids other than CBD, CBG and/or THCV.

According to specific embodiments, the composition is devoid of cannabinoids or cannabis active ingredients other than phytocannabinoids.

According to specific embodiments, the composition or fraction comprises cannabinoids or cannabis active ingredients other than phytocannabinoids.

According to specific embodiments, the composition or fraction comprises at least one terpene or terpenoid.

According to specific embodiments, the composition or fraction comprises at least one of the terpenes and terpenoids listed in Table 1 hereinbelow.

According to specific embodiments, the composition or fraction comprises at least one of the terpenes and terpenoids listed in Table 1 hereinbelow in a concentration ratio according to Table 1±10%.

According to specific embodiments, the composition or fraction comprises at least one of the terpenes and terpenoids listed in Table 1 hereinbelow in a concentration ratio according to Table 1.

According to specific embodiments, the composition or fraction comprises at least two, at least three, at least four, at least five, at least six or all of the terpenes and terpenoids listed in Table 1 hereinbelow.

The compositions of some embodiments of the invention may be synthetic compositions, compositions comprising purified cannabinoids or a fraction of a cannabis extract.

According to specific embodiments, the composition is not a cannabis extract.

According to specific embodiments, the composition is a synthetic composition.

Herein, the term “synthetic composition” refers to a chemically defined composition which can include active ingredients which are chemically synthesized and/or purified to a level of purity of at least 99%.

As used herein “a chemically defined composition” refers to a composition in which all the constituents are known by structure and optionally concentration.

According to specific embodiments, the phytocannabinoids are purified from cannabis.

According to specific embodiments, the phytocannabinoids are synthetic phytocannabinoids.

According to specific embodiments, the composition is cannabis derived fraction.

As used herein “a fraction” refers to a portion of the extract that contains only certain chemical ingredients of the extract but not all.

According to specific embodiments, the composition is a liquid chromatography fraction of a cannabis extract.

According to specific embodiments, the liquid chromatography comprises high pressure liquid chromatography (HPLC) or flash chromatography.

According to specific embodiments, the liquid chromatography fraction of cannabis extract comprises a liquid chromatography pooled fractions of cannabis extract comprising active ingredients detectable by a detector operated at 220 nm, wherein the active ingredients comprise the compounds disclosed herein.

According to specific embodiments, the liquid chromatography fraction is obtainable by subjecting the cannabis extract to flash chromatography comprising a Flash chromatography apparatus equipped with a diode array detector, a C18 functionalized silica column, a 85% methanol in water mobile phase, at a flow rate of 60 ml/min.

According to specific embodiments, the fraction is collected between about 26-31 minutes of the flash chromatography.

According to alternative or additional embodiments, the detector is a diode array detector.

According to alternative or additional embodiments, the detector is a 1260 MWD-VL detector.

Specific embodiments of the present invention also contemplate methods of generating the compositions disclosed herein.

Thus, according to an aspect of the present invention, there is provided a method of generating a composition, the method comprising:
(a) adding a polar solvent to a Cannabis inflorescence so as to obtain a crude extract;
(b) filtering the crude extract so as to obtain a filtered extract;
(c) fractionating the filtered extract on a flash chromatography;
(d) collecting a liquid chromatography fraction(s) detectable by a detector operated at 220 nm and comprising phytocannabinoids, wherein said phytocannabinoids comprise at least 80% CBD and at least one of CBG and THCV.

According to an additional or an alternative aspect of the present invention, there is provided a method of generating a composition capable of reducing dopamine levels secreted from a neuronal cell, the method comprising:
(a) adding a polar solvent to a Cannabis inflorescence so as to obtain a crude extract;
(b) filtering the crude extract so as to obtain a filtered extract;
(c) fractionating the filtered extract on a flash chromatography comprising a Flash chromatography apparatus equipped with a diode array detector, a C18 functionalized silica column, a 85% methanol in water mobile phase, at a flow rate of 60 ml/min;
(d) collecting the liquid chromatography fractions detectable by a detector operated at 220 nm at a retention time between about 26-31 minutes of the flash chromatography.

Cannabis is a genus of flowering plants in the family Cannabaceae that includes three different species, Cannabis sativa, Cannabis indica and Cannabis ruderalis. The term Cannabis encompasses wild type Cannabis and also variants thereof, including cannabis chemovars which naturally contain different amounts of the individual cannabinoids. For example, some Cannabis strains have been selectively bred to produce high or low levels of THC or CBD and other cannabinoids.

According to specific embodiments, the Cannabis plant is a wild-type plant.

According to specific embodiments, the Cannabis plant is transgenic.

According to specific embodiments, the Cannabis plant is genomically edited.

According to specific embodiments, the Cannabis plant is Cannabis sativa (C. sativa).

According to specific embodiments, the Cannabis plant is C. sativa strain Arbel (obtained from IMC, Israeli Medical Cannabis, Israel).

The extract may be derived from a cultivated Cannabis plant (i.e. not grown in their natural habitat) or may be derived from Cannabis plants which grow in the wild.

The tissue of the Cannabis plant from which the extract is typically obtained is the inflorescence. Accordingly, the extract may be obtained from the complete flower head of a plant including stems, stalks, bracts, and flowers. However, it will be appreciated that a cannabis extract of some embodiments the invention may be obtained from only part of the inflorescence, such as from the bracts and/or flowers.

According to specific embodiments, the extract is obtained from a fresh plant (i.e. a plant not heated prior to the extraction process). Fresh plants include plants taken immediately following harvesting (e.g., up to an hour or several hours) for extraction as well as plants frozen immediately after harvesting (e.g. at about −70° C. to −90° C., e.g. at −80° C., for any required length of time) prior to extraction.

According to specific embodiments, the extract is obtained from fresh inflorescence.

According to specific embodiments, the extract is obtained from a frozen inflorescence (e.g. frozen immediately after harvesting at about −70° C. to −90° C., e.g. at −80° C., for any required length of time). Thus, for example, the extract may be obtained from a cryopreserved inflorescence, or from an inflorescence frozen in liquid nitrogen or in dry ice.

According to specific embodiments, the extract is obtained from an inflorescence which has not been subjected to heating (such as heating at e.g. at 120° C. to 180° C., e.g. at 150° C., for any length of time, such as for 1-5 hours).

According to specific embodiments, the extract is obtained from dry Cannabis inflorescence. Drying the inflorescence may be carried out using any method known in the art, such as by pulverizing with liquid nitrogen or with dry-ice/alcohol mixture.

According to specific embodiments, the dry inflorescence is obtained from the grower.

According to specific embodiments, the polar solvent comprises a polar, protic solvent (e.g., ethanol or methanol). In some embodiments, the polar solvent comprises a polar, aprotic solvent (e.g., acetone). Polar solvents suitable for use with specific embodiments of the present invention include, but are not limited to, ethanol, methanol, n-propanol, iso-propanol, a butanol, a pentanol, acetone, methylethylketone, ethylacetate, acetonitrile, tetrahydrofuran, dimethylformamide, dimethylsulfoxide, water, and combinations thereof.

According to specific embodiments, the polar solvent is ethanol (e.g. absolute ethanol i.e. above 99.8%, or in the range of 99-70% in water).

The concentration or amount of a polar solvent used to Cannabis inflorescence can be varied. Generally, the ratio of a Cannabis inflorescence to a polar solvent (weight to volume) is the amount of a polar solvent sufficient to extract about 70% or more, about 75% or more, about 85% or more, about 90% or more, about 95% or more, about 97% or more, or about 99% or more of a composition having a cytotoxic activity. In some embodiments, the ratio of polar solvent to Cannabis inflorescence is about 1:2 to about 1:20 (w/v), e.g. about 1:4 to about 1:10 (w/v).

In particular embodiments, the extract is an ethanol extract.

In particular embodiments, absolute ethanol is added to the inflorescence at a sample-to-absolute ethanol ratio of 1:4 (w/v).

In some embodiments, the Cannabis inflorescence is contacted with a polar solvent (e.g. ethanol) for about 15 minutes or more, about 30 minutes or more, about 1 hour or more, about 2 hours or more, or about 5 hours or more.

According to specific embodiments, the Cannabis inflorescence is contacted with a polar solvent (e.g. ethanol) for about 30 minutes.

Temperature can also be controlled during the contacting. In some embodiments, the Cannabis inflorescence is contacted with a polar solvent at temperature of about 15° C. to about 35° C., or about 20° C. to about 25° C.

According to specific embodiments, the Cannabis inflorescence is contacted with a polar solvent (e.g. ethanol) while being constantly mixed e.g. on a shaker.

In some embodiments, the process of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According to a specific embodiment, the crude extract is filtered through a 0.45-μm syringe filter such as that commercially available from Merck, Darmstadt, Germany.

According specific embodiments, the present invention contemplates drying (i.e. removal of the polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a polar solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment the polar solvent is evaporated with a vacuum evaporator.

According to specific embodiments, the extract (e.g. the filtered extract) is subjected to a decarboxylation step. Decarboxylation may be effected by heating the extract in a pressure tube in the oven at 220° C. for 10 minutes.

Following generation of the filtered extract, specific embodiments of the present invention further contemplate additional purification steps so as to further purify active agents from the extract.

Thus, for example, fractionating the filtered extract. Fractionating can be performed by processes such as, but not limited to: column chromatography, preparative high performance liquid chromatography (“HPLC”), flash chromatography, reduced pressure distillation, and combinations thereof.

According to a specific embodiment, fractionating is performed by HPLC or flash chromatography.

In some embodiments, fractionating comprises re-suspending the filtered extract in a polar solvent (such as methanol, as discussed above), applying the polar extract to a separation column, and isolating the Cannabis fraction by column chromatography (e.g. preparative HPLC, flow cytometry).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N,N-dimethylacetamide, and the like, and combinations thereof.

According to an alternative or an additional embodiment, liquid chromatography is performed on a reverse stationary phase.

According to an alternative or an additional embodiment, liquid chromatography comprises high performance liquid chromatography (HPLC) or flash chromatography, as further described hereinabove and in the Examples section which follows.

The fractions may be characterized by analytical methods such as, but not limited to, spectroscopic methods such as, but not limited to, ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

The fractions obtained may be tested for anti-inflammatory effects on lung epithelial cells and/or pro-inflammatory effects on macrophages. Exemplary methods for testing such effects are further described hereinbelow as well as in the Examples section which follows.

The fractions obtained by the methods may be immediately used or stored until further used.

According to specific embodiments, the fraction is kept frozen, e.g. in a freezer, until further use (e.g. at about −20° C. to −90° C., at about −70° C. to −90° C., e.g. at −80° C.), for any required length of time.

According to other specific embodiments, the fraction is immediately used (e.g. within a few minutes e.g., up to 30 minutes).

The extracts and/or fractions may be used separately. Alternatively, different extracts (e.g. from different plants or from separate extraction procedures) may be pooled together. Likewise, different fractions (from the same extract, from different extracts, from different plants and/or from separate extraction procedures) may be pooled together.

The term “pooled” as used herein refers to collected from the liquid chromatography (e.g. HPLC, flash chromatography) either as a single fraction or a plurality of fractions.

Accordingly, according to an aspect of the present invention, there is provided a composition obtainable by the method.

According to an additional or an alternative aspect of the present invention, there is provided a composition comprising a cannabis derived fraction comprising phytocannabinoids, wherein said phytocannabinoids comprise at least 80% CBD and at least one of CBG and THCV.

The compositions or fractions of some embodiments of the invention have an anti-inflammatory effect on lung epithelial cells.

As used herein the term “anti-inflammatory effect on lung epithelial cells” refers to a significant decrease in inflammation in the presence of the composition or fraction in comparison to same in the absence of the composition or fraction. Such an effect may be manifested by, for example, but not limited to:
(i) reduction of expression and/or secretion of pro-inflammatory cytokines which may be determined by e.g. ELISA, flow cytometry; and/or
(ii) reduction of expression of a receptor associated with the dual renin-angiotensin system (RAS) and/or viral infection which may be determined by e.g. PCR, Western blot, immunostaining.

Non-limiting Examples of pro-inflammatory cytokines which may be used with specific embodiments of the invention include IL-6, IL-8, IL-7, CCL2 and CCL7.

According to a specific embodiment, the decrease is in at least 2%, 5%, 10%, 30%, 40% or even higher say, 50%, 60%, 70%, 80%, 90% or 100%. According to specific embodiments, the decrease is at least 1.5 fold, at least 2 fold, at least 3 fold, at least 5 fold, at least 10 fold, or at least 20 fold as compared to same in the absence of the composition or fraction.

Consequently, according to an aspect of the present invention, there is provided a method of inhibiting inflammation, the method comprising contacting a lung epithelial cell with the composition or fraction disclosed herein.

According to specific embodiments, the contacting is effected in-vitro or ex-vivo.

According to other specific embodiments, the contacting is effected in-vivo.

According to specific embodiments, the method comprises determining inflammation (prior to or following the contacting). Methods of determining inflammation are known in the art and are further described hereinabove and in the Examples section which follows.

According to specific embodiments, the composition or fraction has a combined additive or synergistic anti-inflammatory effect on lung epithelial cells as compared to each of the phytocannabinoids CBD, CBG and THCV when administered as a single agent.

The compositions or fractions of some embodiments of the invention have a pro-inflammatory effect on macrophages.

According to specific embodiments, the synthetic composition does not have a pro-inflammatory effect on macrophages.

As used herein, the term “pro-inflammatory effect on macrophages” refers to a significant increase in macrophage activation in the presence of the composition or fraction in comparison to same in the absence of the composition or fraction.

According to a specific embodiment, the increase is in at least 10%, 30%, 40% or even higher say, 50%, 60%, 70%, 80%, 90%, 100% or higher. According to specific embodiments, the increase is at least 1.5 fold, at least 2 fold, at least 3 fold, at least 5 fold, at least 10 fold, or at least 20 fold as compared to same in the absence of the composition or fraction.

As used herein the term “macrophage activation” or “activating macrophages” refer to the process of stimulating a macrophage that results in cellular maturation, cytokine production, phagocytosis and/or induction of regulatory or effector functions. Methods of determining macrophage activation are well known in the art and include for example the phorbol 12-myristate 13-acetate (PMA) differentiation protocol [see e.g. Starr, T., Bauler, T. J., et al. PLoS ONE 13, e0193601 (2018)].

Such an effect may be manifested by, for example, but not limited to:
(i) macrophage polarization, which may be determined by e.g. microscopy;
(ii) macrophage phagocytosis, which may be determined by e.g. internalization of silica particles;
(iii) expression and/or secretion of pro-inflammatory cytokines, which may be determined by e.g. PCR, western blot, ELISA, flow cytometry; and/or
(iv) expression of receptors that are associated with phagocytosis, which may be determined by e.g. PCR, western blot, immunostaining.

Consequently, according to an aspect of the present invention, there is provided a method of activating a macrophage, the method comprising contacting the macrophage with the composition or fraction disclosed herein.

According to specific embodiments, the method comprises determining macrophage activity (prior to or following the contacting). Methods of determining macrophage activity are known in the art and are further described hereinabove and in the Examples section which follows.

According to specific embodiments, the composition or fraction has a combined additive or synergistic pro-inflammatory effect on macrophages as compared to each of the phytocannabinoids CBD, CBG and THCV when administered as a single agent.

As the fraction of some embodiments disclosed herein is capable of activating macrophages, specific embodiments suggest its use in treating a disease that can benefit from activating macrophages in a subject in need thereof.

Additionally or alternatively, as the synthetic composition of some embodiments disclosed herein is capable of reducing inflammation without activating macrophages, specific embodiments suggest its use in treating cytokine storm in a subject in need thereof.

As used herein, the term “subject” or “subject in need thereof” includes mammals, preferably human beings at any age or gender which suffer from the pathology e.g. disease that can benefit from activating macrophage or cytokine storm. According to specific embodiments, this term encompasses individuals who are at risk to develop the pathology.

According to specific embodiments, the subject is diagnosed with the pathology.

According to specific embodiments, the subject exhibit at least one symptom of the disease.

According to specific embodiments, the subject exhibit at least one symptom of a cytokine storm.

According to specific embodiments, the subject does not have cancer or is not diagnosed with cancer.

As used herein, the term “treating” refers to curing, reversing, attenuating, alleviating, minimizing, suppressing or halting the deleterious effects of a disease or disorder (e.g. disease that can benefit from activating macrophages or cytokine storm). Those of skill in the art will understand that various methodologies and assays can be used to assess the development of a pathology, and similarly, various methodologies and assays may be used to assess the reduction, remission or regression of a pathology.

According to a specific embodiment, treating is preventing.

As used herein, the term “preventing” refers to keeping a disease, disorder or condition from occurring in a subject who may be at risk for the disease, but has not yet been diagnosed as having the disease.

As used herein the phrase “a disease that can benefit from activating macrophages” refers to a disease in which in vivo macrophage activity can at least ameliorate symptoms of the disease or delay onset of symptoms.

Non-limiting examples of diseases that can benefit from activating macrophages include infectious disease, systemic lupus erythematosus, rheumatoid arthritis, multiple sclerosis, a lipid metabolism disorder, atherosclerosis, obstructive jaundice, cholestatic liver disease, skin xanthoma, xanthelasma, tuberous xanthoma, xanthoma striatum, vanishing bile duct syndrome.

According to specific embodiments, the disease is not cancer.

According to specific embodiments, the disease is not in co-morbidity with cancer.

According to specific embodiments, the disease is at a stage prior to cytokine storm.

According to other specific embodiments, the disease is at a stage of cytokine storm.

As used herein, the term “cytokine storm”, also known as “cytokine release syndrome” or “inflammatory cascade”, refers to a disease characterized by dysregulated of pro-inflammatory cytokines, which causes an excessive production of cytokines leading to a positive feedback loop between cytokines and mediator-releasing cells. Non-limiting examples of symptoms of cytokine storm may include high fever, swelling and redness, extreme fatigue and nausea. Cytokine storm may progress to shock, disseminated intravascular coagulation (DIC), and multiple organ failure through neutrophil activation, blood coagulation mechanism activation, and vasodilation.

According to specific embodiments, the disease is an infectious disease, or is associated with infectious disease.

As used herein, the term “associated with infectious disease” means that a pathogen infection leads to the disease.

According to specific embodiments, the infectious disease is not in co-morbidity with other inflammatory diseases.

As used herein, the term “infection” or “infectious disease” refers to a disease induced by a pathogen. Non-limiting specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), intracellular protozoan pathogens (such as, for example, Leishmania and Trypanosoma), parasitic diseases, fungal diseases, prion diseases.

Methods of analyzing infection are well known in the art and are either based on serology, protein markers, or nucleic acid assays.

According to specific embodiments, the infectious disease is associated with a viral infection.

As used herein, the term “associated with a viral infection” means that a viral infection leads to the disease.

Specific types of viral pathogens causing infectious diseases treatable according to specific embodiments of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Non-limiting examples of viral infections include human immunodeficiency virus (HIV)-induced acquired immunodeficiency syndrome (AIDS), coronavirus, influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the viral infection is respiratory viral infection

Non-limiting examples of respiratory viral infections associated with ARDS include a Corona virus infection, a respiratory syncytial virus (RSV) infection, an influenza virus infection, a parainfluenza virus infection, an adenovirus infection and a rhinovirus infection.

According to specific embodiments, the viral infection is a Corona virus infection.

According to specific embodiments, a clinical manifestation of Corona virus infection includes symptoms selected from the group consisting of inflammation in the lung, alveolar damage, ARDS, fever, cough, shortness of breath, diarrhea, organ failure, pneumonia, cytokine storm, septic shock and/or blood clots.

As used herein, “Corona virus” refers to enveloped positive-stranded RNA viruses that belong to the family Coronaviridae and the order Nidovirales.

Examples of Corona viruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, Corona viruses, which are zoonotic in origin, can evolve into a strain that can infect human beings leading to fatal illness. Thus particular examples of Corona viruses contemplated herein are SARS-CoV, Middle East respiratory syndrome Coronavirus (MERS-CoV), and the recently identified SARS-CoV-2 [causing 2019-nCoV (also referred to as “COVID-19”)].

It would be appreciated that any Corona virus strain is contemplated herein even though SARS-CoV-2 is emphasized in a detailed manner.

According to specific embodiments, the Corona virus is SARS-CoV-2.

As used herein the SARS-CoV-2 includes any variants and mutants thereof including, but not limited to, the B.1.1.7 (Alpha), B.1.351 (Beta), B.1.617.2 (Delta), P.1 (Gamma), B.1.526 (Iota), B.1.427 (Epsilon), B.1.429 (Epsilon), B.1.617 (Kappa, Delta), B.1.525 (Eta) and P.2 (Zeta).

According to specific embodiments, the composition or fraction can be used alone or in combination with other established or experimental therapeutic/prophylactic regimen to the disease. Hence, according to specific embodiments, the compositions disclosed herein are provided to the individual with additional active agents to achieve an improved therapeutic or preventive effect as compared to treatment with each agent by itself. Thus, for example, for treating a Corona virus infection the compositions may be administered in conjunction with e.g. mechanical ventilation, neuromuscular blockers, extracorporeal membrane oxygenation (ECMO), anti-viral drug, anti-fungal drug, anti-bacterial drug, immune-globulin treatment, glucocorticoid therapy, vaccine. In such therapy, measures (e.g., dosing and selection of the complementary agent) are taken to prevent adverse side effects which may be associated with combination therapies.

According to a specific embodiment, the composition or fraction is not used in combination with a non-steroidal anti-inflammatory drug (NSAID).

According to a specific embodiment, the composition or fraction is not used in combination with a steorid.

Each of the compositions or fractions described herein can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cannabis derived active ingredients e.g. phytocannabinoids accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as an oil-based formulation, tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

The pharmaceutical composition can be formulated for inhalation. For example, the compositions can be formulated as vapors or aerosols that can be inhaled into the lungs. Vapor formulations include liquid formulations that are vaporized when loaded into a suitable vaporization device.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For transdermal administration, the composition can be formulated in a form of a gel, a cream, an ointment, a paste, a lotion, a milk, a suspension, an aerosol, a spray, a foam, a serum, a swab, a pledget, a pad or a patch. Formulations for transdermal delivery can typically include carriers such as water, liquid alcohols, liquid glycols, liquid polyalkylene glycols, liquid esters, liquid amides, liquid protein hydrolysates, liquid alkylated protein hydrolysates, liquid lanolin, lanolin derivatives, glycerin, mineral oil, silicone, petroleum jelly, lanolin, fatty acids, vegetable oils, parabens, waxes, and like materials commonly employed in topical compositions. Various additives, known to those skilled in the art, may be included in the transdermal formulations of the invention. For example, solvents may be used to solubilize certain active ingredients substances. Other optional additives include skin permeation enhancers, opacifiers, anti-oxidants, gelling agents, thickening agents, stabilizers, and the like.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (cannabis derived active ingredients) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

A non-limiting example of an animal model for SARS-CoV-2 is the transgenic mouse expressing human ACE2 (see e.g, Bao et al. (2020) Nature 583: 830-833.

The doses determined in the mouse animal model can be converted for the treatment other species such as human and other animals diagnosed with the disease, using conversion Tables known to the skilled in the art.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The compositions disclosed herein can be administered to a subject (e.g., a human) in need thereof in a variety of other forms including a nutraceutical composition.

As used herein, a “nutraceutical composition” refers to any substance that may be considered a food or part of a food and provides medical or health benefits, including the prevention and treatment of disease. In some embodiments, a nutraceutical composition is intended to supplement the diet and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

Non-limiting forms of nutraceutical compositions of the present invention include: a tablet, a capsule, a pill, a softgel, a gelcap, a liquid, a powder, a solution, a tincture, a suspension, a syrup, or other forms known to persons of skill in the art. A nutraceutical composition can also be in the form of a food, such as, but not limited to, a food bar, a beverage, a food gel, a food additive/supplement, a powder, a syrup, and combinations thereof.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Extract preparation—High CBD Cannabis sativa strain Arbel (IMC, Israel) dry inflorescence was extracted using ethanol as previously described³⁸, and decarboxylated by heating the dried crude extract to 220° C. for 10 minutes. The dried decarboxylated extract was weighed, and then resuspended in absolute methanol (volume of solvent added according to the desired concentration) and filtered through a 0.45 μm syringe filter.

Extract fractionation—A flash chromatography apparatus (Flash Pure, Buchi, C-810) equipped with a diode array detector was used to fractionize the decarboxylases crude extract. An Ecoflex C-18 80 g, 50 μm spherical, max. pressure 180 psi column was used for separation, with 85% methanol in water as the mobile phase. The flow rate was 60 mL/min. The retention time for F_{CBD} was between 26-31 minutes. The organic solvent (methanol) of each fraction was removed by using rotary vacuum evaporator at 30° C. The remaining aqueous phase containing compound of interest was further lyophilized for getting dried powder. Each dried fraction tube was separately weighed and reconstituted by methanol to produce a required concentration and stored in −20° C.

Chemical analyses—High performance liquid chromatography (HPLC 1260 Infinity II, Agilent) equipped with Raptor ARC-18 for LC-UV column (150 mm×4.6 mm ID, pore size 2.7 μm) was used for chemical analysis as previously described³⁸ using isocratic separation with acetonitrile (25%) and water with 5 mM ammonium formate, 0.1% formic acid (75%) at a constant flow rate of 1.5 mL/min. Sample concentration was 100 μg/mL and injected volume was 5 μL. Cannabinoids profile and quantification were done in comparison to standard calibration curves obtained by dissolving cannabinoids standards in methanol at concentrations range of 0-50 μg/mL. Gas chromatography-mass spectrometry (GC8860-MS5977B Agilent) equipped with 30 m, 0.25 mm ID, 5% cross-linked phenylmethyl siloxane capillary column (HP-5MS) with 0.25-μm film thickness used for chemical analysis as described³⁸. 10 μL of each fraction were transferred into GC vial with an insert, dried under a gentle stream of nitrogen and dissolve in 100 μL of Hexane. Sample volume for injection was 1 μL. Helium was used as the carrier gas at a constant flow of 1.1 mL s-1. An isothermal hold at 50° C. was maintained for 2 minutes, followed by a heating gradient of 6° C. min-1 to 300° C., and the final temperature was held for 4 minutes. Peak assignments were performed with spectral libraries (NIST 17.0 and 14.0) and compared with MS data obtained from the injection of standards purchased from LGC Standards.

Standard/material preparation—The cannabinoid standards at a concentration of 1 mg/mL in methanol included cannabidiol (CBD, Restek catalog no. 34011) cannabigerol (CBG, Restek catalog no. 34091) and tetrahydrocannabivarin (THCV, Restek catalog no. 34100). Inverse agonists (IA) to cannabinoid receptors type 1 (CB1) and 2 (CB2) used were Abcam products: CB1 (AM251, ab120088), CB2 (SR144528, ab146185) and TRPA1 blocker (HC-030031, ab120554), all dissolved in DMSO at a concentration of 10 mM. Phorbol 12-myristate 13-acetate (PMA) (P1585; Sigma Aldrich, USA) was dissolved in DMSO at the stock concentration of 5 μg/mL. Dexamethozone (D4902, Sigma Aldrich, USA) was dissolved in methanol at the stock concentration of 1000 μg/mL. Ruxolitinib JAKAVI was dissolved in DMSO at the concentration of 5000 μg/mL, confirmed with GCMS and HPLC. TNFα (300-01A; PeproTech, Rocky Hill, N.J., USA) was dissolved in water at the stock concentration of 100 μg/mL. (3-Aminopropyl) triethoxysilane (APTES), N-(3-Dimethylaminopropyl)-N(3-ethylcarbodiimide hydrochloride (EDC), and 5(6)-Carboxyfluorescein, 2-(4-Morpholino) ethanesulfonic acid (MES) were purchased from Sigma-Aldrich (USA). Analytical grade methanol and ultra-pure deionized water (MS grade) were used as received without further purification. Palmitic acid (Sigma Aldrich; P0500, USA) was dissolved in methanol at the stock concentration of 0.5 mol/L and used at 150 μM. Alamar Blue (Resazurin AR002, R&D Systems, Minneapolis, Minn., USA), was used according to manufacturer's instructions.

Cell cultures—The lung cancer cell line A549 (ATCC® CCL-185™) was cultured in DMEM (01-055-1A, Biological Industries, Israel) growth media supplemented with 10% FBS (04-127-1A, Biological Industries, Israel), 1% Glutamic acid, 1% pen-strep and plasmocin. Macrophage cell line KG1 (ATCC® CCL-246™) was cultured in IMDM (01-058-1A; Biological Industries, Israel) containing 20% FBS and 1% pen-strep and plasmocin. IMDM media supplemented with 10 ng/mL PMA, 5% FBS, 1% pen-strep and plasmocin was used as stimulating environment for the differentiation of KG1 cells. Differentiated cells with typical morphology were attached to the plate surface within 1-2 days of initiation³⁹.

Determination of IL levels and cell viability—IL-6 and IL-8 levels were determined as described previously⁴⁰ with the following modifications: A549 cells were plated at 5×10⁴ cells per well in DMEM complete media (400 μL) in a 24-well cell culture plate. Cells were allowed to attach and incubated with complete DMEM at 37° C. in a humidified atmosphere containing 5% CO₂-95% air. Following overnight incubation, cells were treated with 300 ng/mL TNFα with or without plant extract or fraction/compounds. IL-6 and IL-8 secretion levels were analyzed after 4 hours of incubation. Supernatant samples were taken and tested using IL-6 and IL-8 ELISA kits (DY206 and DY208 respectively, R&D Systems, Minneapolis, Minn., USA). Dexamethasone was used as a positive control. For cell viability, an Alamar Blue (resazurin) assay was performed on each well as described previously⁴⁰. For dose response assays, data points were connected by non-linear regression lines of the sigmoidal dose-response relation. GraphPad Prism (GraphPad Software Inc., San Diego, USA) was employed to produce dose-response curves and IC₅₀ doses were calculated using nonlinear regression analysis.

Salinization of silicon dioxide surfaces with APTES—To prepare the silica dispersion, 1 g of silica was added to 40 mL of methanol and stirred. Following, APTES (0.0045 moles) was slowly added to the solution. The reaction was carried out at ambient temperature for 45 minutes. Following silanization, 50-100 nm or 30-70 nm particles were collected by centrifugation (9000 rpm, 10 minutes) washed 4 times with water, and dried at 35° C. under vacuum for 3 hours 41.

Labeling of amine functionalized silica nanoparticles with 5(6)-Carboxyfluorescein and IgG—Stock solutions of 1 mg of EDC were prepared separately in 1 mL of 0.1 M MES (pH 4.5-5) buffer. 100 mg of the amine functionalized silica nanoparticles were added to 600 μL of the MES buffer followed by 200 μL of the EDC. The mixture was vortexed for 10 minutes. Following, 100 μL 5(6)-Carboxyfluorescein (1 mg/mL) only or 50-100 nm SNP or 30-70 nm ENP nanoparticles or 100 μL 5(6)-Carboxyfluorescein (1 mg/mL) and IgG (10 mg/mL; for 30-70 nm ENPG nanoparticles) was added. The final solution was then mixed by vortex for 3 hours at ambient room temperature. Subsequently, the mixture was centrifuged and rinsed with MES buffer to remove excess reactants. EDC was used as a cross-linker to chemically attach the carboxyl group of the 5(6)-Carboxyfluorescein molecule and producing an amine-reactive O-acylisourea. For the Fluorescent-IgG labelled silica nanoparticles this intermediate product reacted with the amino groups of the silica nanoparticles to yield an amide bond, releasing fluorescent-IgG labelled silica nanoparticles and urea as a by-product⁴². The fluorescent labelled (SNP or ENP) or Fluorescent-IgG labelled (ENPG) silica nanoparticles were then dispersed again in the MES buffer for further analysis.

Cellular staining and confocal microscopy—Differentiated macrophages from KG1 cells (10×10⁴ cells/plate; plated on the bottom of a glass cell culture dish) were incubated in 500 μL of 5% FBS-IMDM media with SNP, ENP or ENPG (40 μg/mL) and incubated for 4 hours at 37° C. for phagocytosis. Macrophages that underwent phagocytosis were fixed with 3.7% formaldehyde solution and permeabilized with 0.1% Triton X-100 at room temperature. Fixed cells were blocked in PBS containing 1% bovine serum albumin. Cells were incubated with EasyProbes™ ActinRed 555 Stain for actin and Hoechst for nuclear staining (AP-FP032, GC-C057 respectively; ABP Bioscience Rockville, Md., USA). Cell microscopy and image acquisition was carried out using a Leica SP8 laser scanning microscope (Leica, Wetzlar, Germany), equipped with a 405, 488 and 552 nm solid state lasers, HCX PL APO CS 10×/0.40 or HC PL APO CS 63×/1.2 water immersion objective (Leica, Wetzlar, Germany) and Leica Application Suite X software (LASX, Leica, Wetzlar, Germany). Hoechst, 5(6)-Carboxyfluorescein and ActinRed 555 emission signals were detected with PMT and HyD (hybrid) detectors in ranges of 415-490 nm, 500-535 nm and 565-660 nm, respectively.

Quantitative real-time (qRT) PCR—qRT PCR was effected as described³⁸. Briefly, cells were seeded in a 6-well plate at a concentration of 1.5×10⁶ cells in 3 mL per well. Following 24 hours incubation, cells were treated with extract/fraction/compounds for 6 hours. Cells were harvested and RNA was extracted using TRI reagent (T9424, Sigma Aldrich, USA). RNA was reverse-transcribed in a total volume of 20 μL (PB30.11-10; qPCRBIO), according to manufacturer's protocol. PCR was performed in triplicate using qPCR SyGreen Blue Mix (PB20.16-20, qPCRBIO) and a StepOnePlus system (Applied Biosystems). The expression of each target gene was normalized to the expression of HPRT1 mRNA in the 2^{-ΔΔct} method, presenting the differences (Δ) in threshold cycle (Ct) between the target gene and HPRT1 gene. ΔCt=Ct Target gene—Ct HPRT1. ΔΔCt=ΔCt treatment-ΔCt control. Experiments were repeated three times. The following primers were used:

ACE2 (Gene ID: 59272): forward
(SEQ ID NOs: 1-2)
5′-AAGCACTCACGATTGTTGGG-3′
and
reverse
5′-CACCCCAACTATCTCTCGCT-3′;
CCL2 (Gene ID: 6347): forward
(SEQ ID NOs: 3-4)
5′-AAGGAGATCTGTGCTGACCC-3′
and
reverse
5′-GCTGCAGATTCTTGGGTTGT-3′;
IL-6 (Gene ID: 3569): forward
(SEQ ID NOs: 5-6)
5′-GAACTCCTTCTCCACAAGCG-3′
and
reverse
5′-GAAGAGGTGAGTGGCTGTCT-3′;
CCL7 (Gene ID: 6354): forward
(SEQ ID NOs: 7-8)
5′-CACCCTCCAACATGAAAGCC-3′
and
reverse
5′-GGTGGTCCTTCTGTAGCTCT-3′;
IL-7 (Gene ID: 3574): forward
(SEQ ID NOs: 9-10)
5′-CTGAAAGTACACTGCTGGCG-3′
and
reverse
5′-GAGTTGCCGAGTCTGTGTTG3′;
FCγR2A (Gene ID: 2212): forward
(SEQ ID NOs 11-12)
5′-GCCAATTCCACTGATCCTGT-3′
and
reverse
5′-CCTGGGGTTCAGAGTCATGT-3′;
SCARBI (Gene ID: 949): forward
(SEQ ID NOs: 13-14)
5′-CTGTGGGTGAGATCATGTGG-3′
and
reverse
5′-GTTCCACTTGTCCACGAGGT-3′;
CD36 (Gene ID: 948): forward
(SEQ ID NOs: 15-16)
5′-AGATGCAGCCTCATTTCCAC-3′
and
reverse
5′-TGGGTTTTCAACTGGAGAGG-3′;
IL-8 (Gene ID: 3576): forward
(SEQ ID NOs: 17-18)
5′-CAGGAATTGAATGGGTTTGC-3′
and
reverse
5′-AAACCAAGGCACAGTGGAAC-3′.

Imaging Flow Cytometry—Differentiated macrophages from KG1 cells (10×10⁵ cells/well) were seeded in a 6-well plate culture dishin 2 mL of 5% FBS-IMDM media containing SNP, ENP, or ENPG (40 μg/mL) and incubated for 4 hours at 37° C. for phagocytosis. The cells were detached from the surface of the plate using a trypsin 0.25%:EDTA 0.05% solution (03-052-1A, Biological Industries, Israel) for 3 minutes, washed with DMEM complete media, centrifuged and transferred to 50 μL cold PBS kept on ice. Cells were analyzed by multispectral imaging flow cytometry (ImageStream markII flow cytometer; Amnis Corp, part of EMD Millipore, Seattle, Wash., USA). Fluorescence intensity of the Fluorescein labeled silica beads was measured in channel 2 of the cytometer (480 nm ex, 560 nm em). A ×60 magnification was used for all samples. At least 4,000 cells were collected for each sample and data was analyzed using a dedicated image analysis software (IDEAS 6.2; AmnisCorp). Cells were gated for single cells using the area and aspect ratio features, and for focused cells using the Gradient RMS feature. Cropped cells were further eliminated by plotting the cell area of the bright field image against the Centroid X feature (the number of pixels in the horizontal axis from the left corner of the image to the center of the cell mask). Cells were further gated for cells that were positive for beads (for ENP or ENPG), or for cells with SNP on their surface or inside the cells, using the intensity feature (the sum of the background—subtracted pixel values within the masked area of the image) and max pixel (the largest value of the background subtracted pixel). SNP internalization was calculated by the internalization feature, i.e. the ratio of the intensity inside the cell to the intensity of the entire cell, mapped to a log scale. To define the internal mask for the cell, the object mask of the brightfield image was eroded by 8 pixels. Cells with an internalization score higher than 0.33 were gated as cells with internalized SNP.

Statistical analysis—The data was processed using JMP statistical package (SAS Inc, NC, USA). Comparisons between two groups were made with the Student's T-Test. Comparisons between more than 2 groups were made with two-way analysis of variance (ANOVA) followed by Tukey-Kramer's honest significant difference (HSD) test as post hoc. Values are shown as mean±standard error (s.e.m.). P values≤0.05 were considered significant.

### Example 1

C. sativa Extracts, Chromatography Fraciotnated Fractions and Synthetic Composition Mimicking Same have Anti-Inflammatory Activity on Lung Epithelial Cells

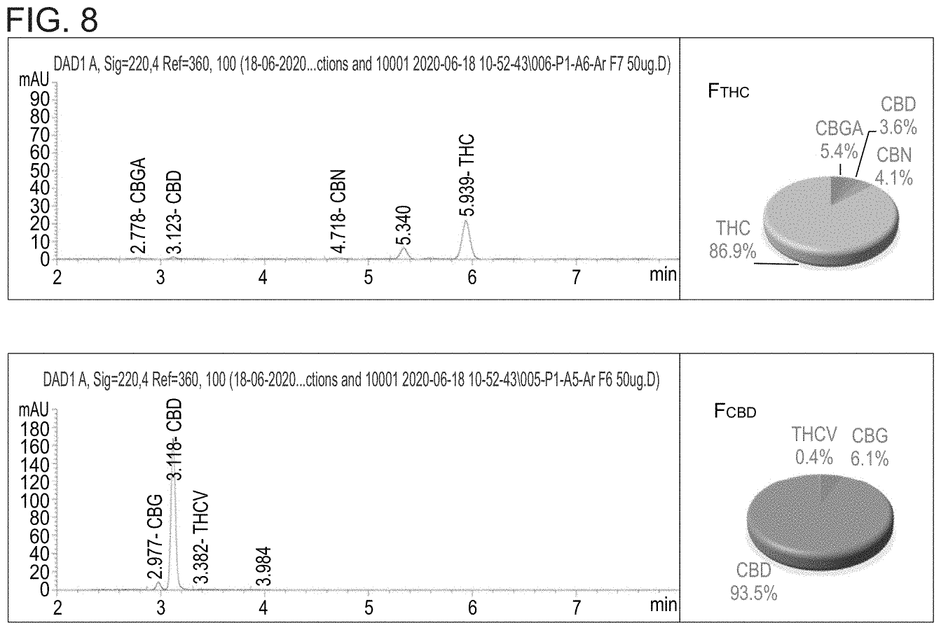

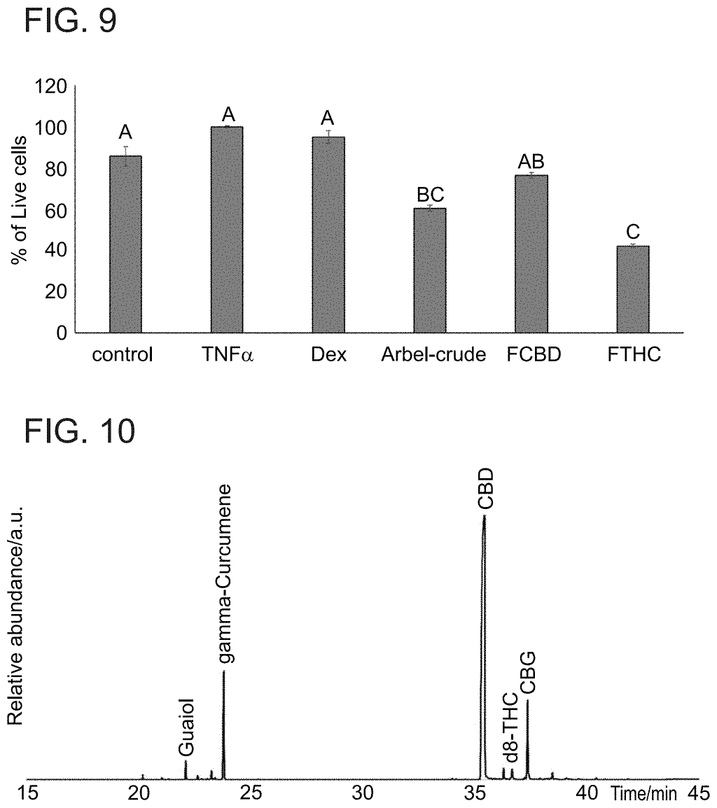

The high CBD cannabis strain Arbel was used to examine extract activity in reducing inflammation induced by TNFα in the lung epithelial cancer cell line A549. The crude extract at 5 μg/mL led to a substantial reduction in the levels of secreted IL-6 and IL-8 (FIGS. 1A-B). Subsequently, chromatography fractionated fractions referred to herein as high CBD (F_{CBD}) and high THC (F_{THC}) fractions (FIG. 8) were examined for their anti-inflammatory activity (FIGS. 1A-D). Fraction F_{THC} exhibited only low anti-inflammatory activity; however, fraction F_{CBD} significantly reduced IL-6 and IL-8 secretion levels from lung epithelial cells, with IC₅₀ of 3.45 and 3.49 μg/mL, respectively. F_{CBD} activity was even greater than that of dexamethasone at 4 μg/mL in reducing IL-8 levels (FIGS. 1A-B). In addition, while F_{CBD} anti-inflammatory activity was similar to that of the crude extract for reducing both IL-6 and IL-8 levels, the crude extract (and F_{THC}) led to substantial cell death, whereas F_{CBD} at 5/mL was comparatively less cytotoxic (76.7% viability; FIGS. 1A-B and 9).

Based on HPLC and GC/MS analysis, F_{CBD} contains approximately 66% phytocannabinoid by total content. The phytocannabinoid assemblage included CBD (93.5%), CBG (6.1%) and minute amount of THCV (0.4%) (FIG. 8). Multiple terpenes were detected in F_{CBD} (Table 1 hereinbelow and FIG. 10). A synthetic composition containing combination of phytocannabinoid standards at the ratios found in fraction F_{CBD} (referred to herein as F_{CBD:std}) had a similar effect on IL-6 and IL-8 secretion compared to the chromatography fractionated fractions (IC₅₀ of 4.1 μg/mL for IL-6 and IL-8; FIGS. 1E-F).

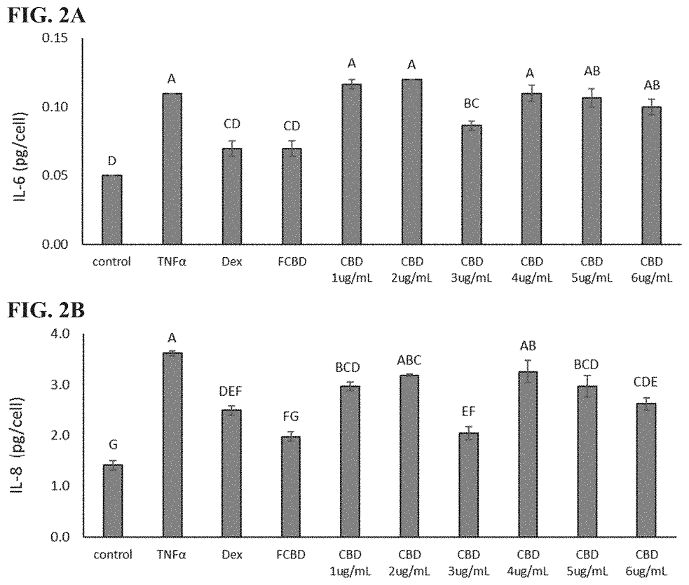

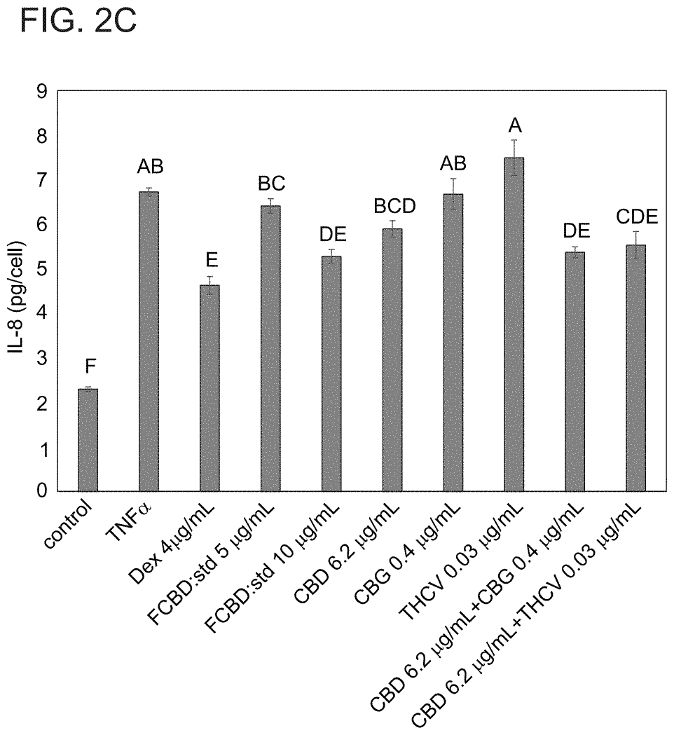

Interestingly, CBD (the main phytocannabinoid in F_{CBD}) alone showed a bell shaped activity curve, i.e., 3.0 μg/mL showed anti-inflammatory activity for both IL-6 and IL-8 levels, similar to F_{CBD} at 4.1 μg/mL (FIGS. 2A-B). Nevertheless, higher or lower concentrations of CBD had lower and/or non-significant activity in reducing IL-6 and IL-8 levels (FIGS. 2A-B).

Further, synthetic compositions containing combination of CBD and CBG or CBD and THCV at the concentration and ratio found in 10 μg/ml F_{CBD:std} decreased IL-8 and IL-6 secretion while CBD, CBG or THCV administered as single agents at their concentrations in 10/ml F_{CBD:std} did not lead to such a reduction (FIG. 2C).

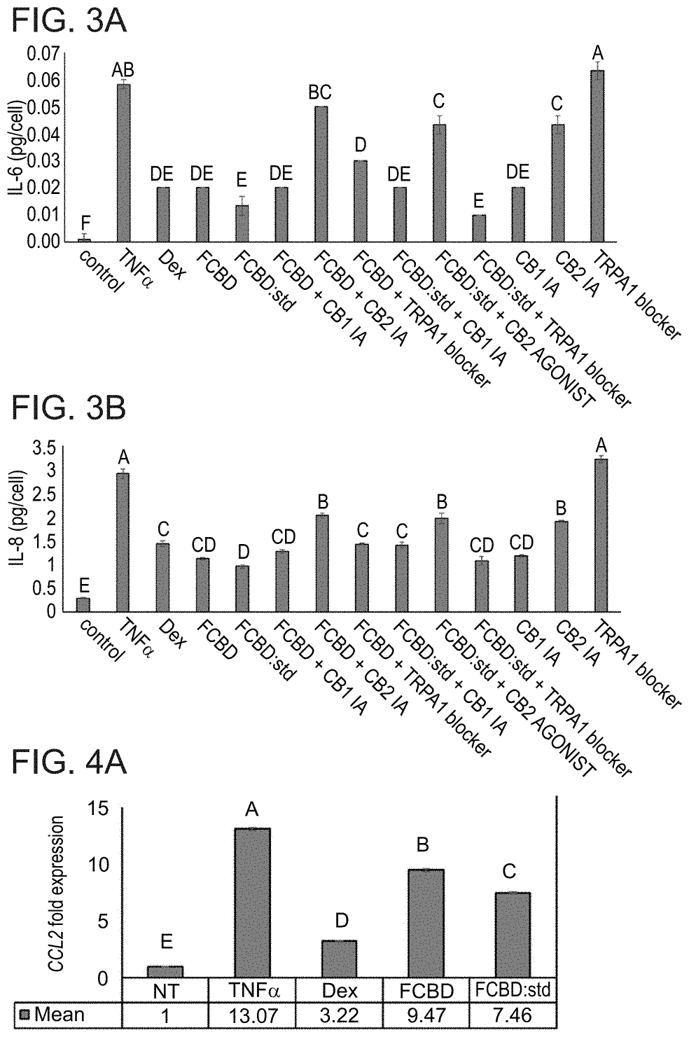

Using CB2 receptor inverse agonists (IA) with F_{CBD} or F_{CBD:std} treatments reduced the effect of the fraction and standard mix on IL-6 and IL-8 secretion from A549 cells (FIG. 3A-B). However, treatment with CB1 IA or a TRPA1 blocker did not affect F_{CBD} or F_{CBD:std} activity. Treatment with CB1 or CB2 IA led only to reduction in IL-6 and IL-8 levels in these cells, CB1 to a greater extent (FIGS. 3A-B).

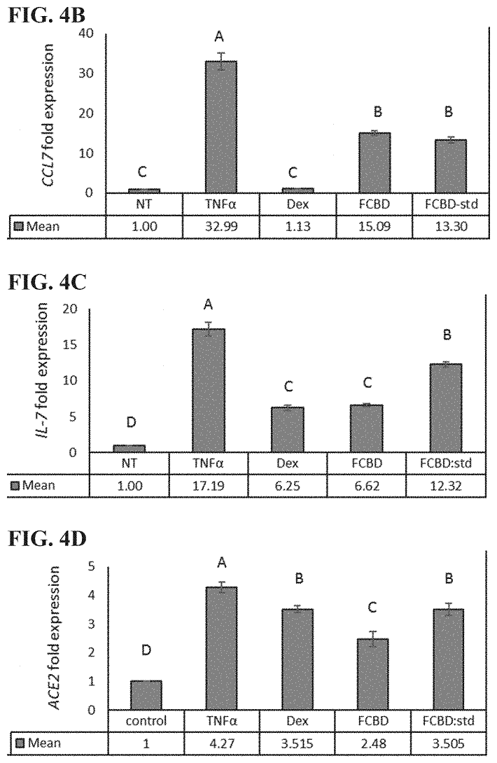

qPCR analysis demonstrated that F_{CBD} or F_{CBD:std} treatments reduced the mRNA steady state level of the pro-inflammatory cytokines CCL2 and CCL7 in TNFα treated A549 cells (using HPRT1 as a reference gene) (FIGS. 4A-B). However, the reduction in expression of the two genes was less than that of dexamethasone (FIGS. 4A-B). F_{CBD:std} treatment led to a minor though significant reduction in the expression level of IL-7, whereas F_{CBD} and dexamethasone substantially reduced IL-7 expression (FIG. 4C). Moreover, F_{CBD} or F_{CBD:std} treatments reduced the expression level of ACE2, F_{CBD} to a greater extent than dexamethasone or F_{CBD:std} (FIG. 4D). The ACE2 receptor is a part of the dual renin-angiotensin system (RAS) [Dalan, R.; et al. Horm. Metab. Res. 52, 257 (2020).] and was shown to be involved with SARS-CoV-2 human infection [Li, F., et al. Science 309, 1864-1868 (2005).]. Specifically, in cells of patients with severe symptoms of COVID-19, ACE2 was substantially upregulated 199 fold; this upregulation was suggested to be one of the factors leading to disruption of the RAS, as ACE2 is a part of the counteracting hypotensive axis of RAS. The increase in ACE2 and other key RAS components is predicted to elevate bradykinin levels in multiple tissues, leading to increases in vascular permeability and hypotension; the latter is highly associated with severe COVID-19 patients [Garvin, M. R. et al. elife 9, e59177 (2020).]. Indeed, a negative correlation was identified between ACE2 gene expression and COVID-19 mortality [Chen, J. et al. Aging Cell 19, e13168 (2020).].

TABLE 1
Chemical composition of terpenes and terpenoids
in fraction F_{CBD} analyzed using gas chromatography
coupled with mass spectrometer (GC-MS)

| Compound | % of terpenes | % of total |
|---|---|---|
| Butylated hydroxytoluene | 2.6 | 0.3 |
| 1,6-Dioxacyclododecane-7,12-dione | 1 | 0.1 |
| Guaiol | 10.4 | 1.2 |
| γ-Eudesmol | 2.3 | 0.3 |
| α-Eudesmol | 5.6 | 0.6 |
| Guaienol | 1.3 | 0.2 |
| γ-Curcumene | 75.6 | 8.7 |
| other | 1.2 | 0.1 |

Taken together, F_{CBD} and phytocannabinoid standards that mimic same has a significant anti-inflammatory activity on alveolar epithelial cells. Moreover, a combination of CBD with CBG and/or THCV in a concentration and ratio mimicking F_{CBD} has a higher anti-inflammatory effect as compared to treatment with each of the phytocannabinoids administered as a single agent.

### Example 2

C. sativa Chromatography Fraciotnated Fraction F_{CBD} and a Synthetic Composition Mimicking Same Affect Macrophage Activity

F_{CBD} and F_{CBD:std} Treatments Induce IL-6, IL-8 and CCL2 Expression in Differentiated Macrophages

During the second phase of COVID-19, pneumonia patients exhibit features of macrophage activation syndrome (MAS) in which macrophages play a major pro-inflammatory role by releasing pro-inflammatory cytokines such as IL-6, IL-8 and CCL2 [Ruscitti, P., et al. Autoimmun. Rev. 19, 102562 (2020).]. Moreover, subsets of macrophages in patients with COVID-19 were found to express genes associated with IL-6, whereas expression of IL-6 was again associated with severe depletion of lymphocytes from the spleen and lymph nodes [Merad, M. & Martin, J. C. Nat. Rev. Immunol. 20, 355-362 (2020)]. F_{CBD} treatment increased IL-6, IL-8 and CCL2 mRNA expression levels in PMA-treated KG1 cells (differentiated macrophages) by ˜2, ˜433 and ˜49 fold, respectively (FIGS. 5A-C). F_{CBD:std} increased CCL2 mRNA expression by −20 fold (FIG. 5C) and IL-8 mRNA expression level by −26 fold; however F_{CBD:std} did not lead to an increase in IL-6 mRNA expression level in macrophages (FIGS. 5A-C). At the protein level, F_{CBD} but not F_{CBD:std} increased IL-8 secretion from KG1 cells treated with TNFα in a dose dependent manner (FIGS. 5D-E). Dexamethasone (at 8 or 4 μg/mL) had no effect on macrophages IL-6, IL-8 and CCL2 mRNA expression levels or on IL-8 secretion from macrophages (FIGS. 5A-E).

F_{CBD} and F_{CBD:std} Attenuate Macrophages Polarization

To examine the effect of the treatments on macrophage polarization, PMA-treated KG1 cells were incubated with FNP. In the control (vehicle treated), most of the cells were non-polarized and featured a round structure, whereas the macrophage population treated for 16 hours with F_{CBD} (7 μg/mL) contained ˜48% polarized cells (Table 2 hereinbelow; FIG. 6). Further, multiple silica particles and membrane pseudopods were detected in these polarized cells (FIG. 6). Likewise, treatment of the macrophage population with F_{CBD:std} resulted in ˜49% polarized cells (Table 2 hereinbelow). Accordingly, lower concentrations of F_{CBD} (3.5 μg/mL) led to a somewhat reduced percentage of polarized cells (˜45%) and treatment of macrophages with CBD at the equivalent concentration (i.e. 4.35 μg/mL) as in F_{CBD} at a concentration of 7 μg/mL resulted in only −18% polarized cells (Table 2 hereinbelow).

TABLE 2
Percentage of polarized cells out of total cells of differentiated
KG1 cell population that were counted (n = 5).

| | | Total number of cells |
|---|---|---|
| | | counted in all |
| Treatment | % of polarized cells | replicates (n = 5) |
| Vehicle Control | 1.2 ± 0.83^{b} | 204 |
| F_{CBD} (7 μg/mL) | 48.3 ± 6.9^{a}* | 144 |
| F_{CBD-std} (7 μg/mL) | 48.8 ± 11.3^{a}* | 74 |
| CBD (4.3 μg/mL) | 17.9 ± 4.1^{ab}* | 94 |
| F_{CBD} (3.5 μg/mL) | 44.9 ± 12.4^{a}* | 70 |

Means with different letters are significantly different from all combinations of pairs by Tukey-Kramer honest significant difference (HSD; P ≤ 0.05).
*Mean significantly different from control based on Student T-test (P ≤ 0.05).

F_{CBD} Attenuates Expression of Phagocytosis-Associated Receptors

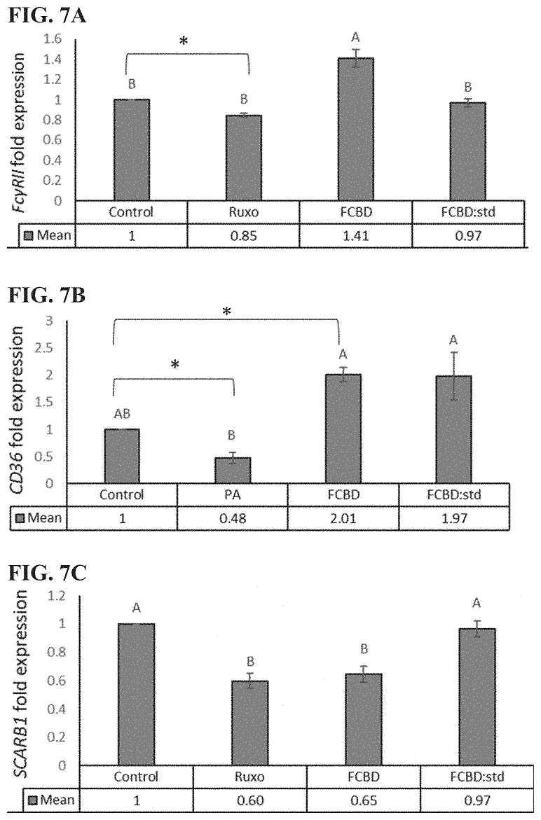

SCARB1 encodes SR-B1 that is a scavenger receptor (class B) and is also responsible for phagocytosis of silica particles in macrophages F_{CBD} reduced expression of SCARB1; SCARB1 encodes SR-B1 that is a scavenger receptor (class B) and is also responsible forphagocytosis of silica particles in macrophages [Tsugita, M., et al. Cell Rep. 18, 1298-1311 (2017).]. Phagocytosis is initiated by the ligation of Fcγ receptors to IgG-opsonins on the target cell [Greenberg, S. & Grinstein, S. Curr. Opin. Immunol. 14, 136-145 (2002).], whereas CD36 expression in macrophages was shown to be involved with lung fibrosisin in mice [Wang, X., Lv, L., et al. Toxicol. Ind. Health 26, 47-53 (2010).]. F_{CBD} treatment, but not F_{CBD}:std, increased mRNA expression of FcγRII and CD36, in comparison to the vehicle control (FIGS. 7A-B). Treatment with Ruxolitinib which inhibits monocyte activation¹² reduced FcγRII expression (FIG. 7A), and PA reduced expression of CD36 (FIG. 7B) in agreement with¹³. In addition, mRNA expression of SCARB1 was reduced by F_{CBD} and Roxulitinib but not by F_{CBD:std} (FIG. 7C).

F_{CBD} Increase Internalization of Silica Particles in Macrophages

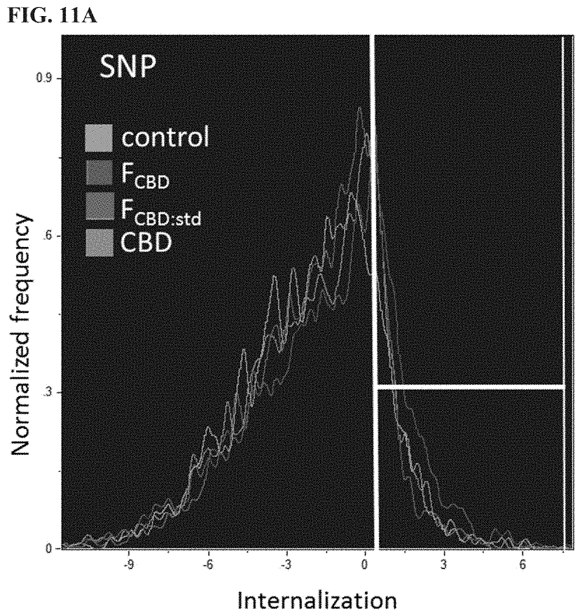

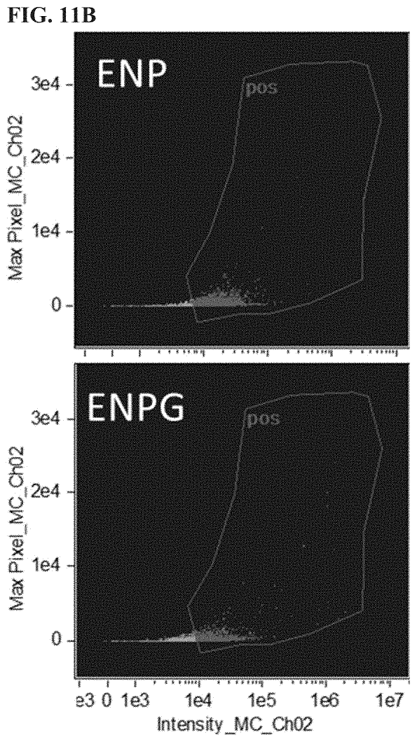

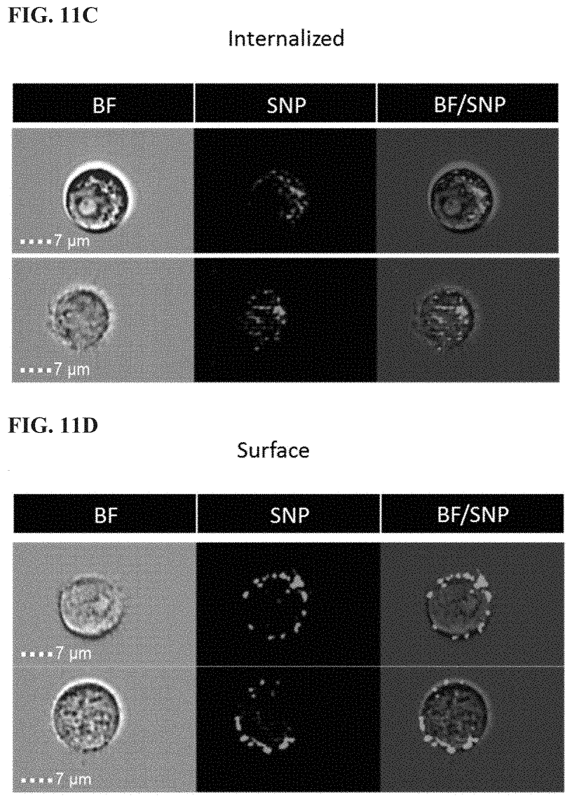

Based on cell analysis by Imaging Flow Cytometry for macrophages with internalized silica particles, it was found that F_{CBD} increased the percentage of cells that internalized fluorescent-labeled silica particles (SNP, Table 3 hereinbelow; FIGS. 11A-D). The increase in percentage of positive cells by F_{CBD} was higher in comparison to the vehicle control also for the smaller fluorescent-labeled silica particles, ENP and ENPG. F_{CBD:std} and CBD treatments were less effective in increasing of internalization (for SNP) or presence (ENP and ENPG) of the particles in cells in comparison to the F_{CBD} treatment (Table 3 hereinbelow; FIGS. 11A-D).

TABLE 3
Percentage of macrophage cells with internalization of SNP silica
beads or positive for ENP or ENPG silica beads analyzed using
Imaging Flow Cytometry following the indicated treatment.

| Treatment | ENPG | ENP | SNP |
|---|---|---|---|
| Vehicle control | 100.0^{a} | 100.0^{a} | 100.0^{b} |
| F_{CBD} (7 μg/mL) | 132.9 ± 30.3^{a} | 167.9 ± 11.2^{a} | 147.8 ± 13.4^{a} |
| F_{CBD: std} (7 μg/mL) | 116.2 ± 3.1^{a} | 125.3 ± 10.2^{a} | 99.8 ± 0.8^{b} |
| CBD (4.35 μg/mL) | 89.6 ± 3.9^{a} | 121.3 ± 24.0^{a} | 118.85 ± 5.10^{ab} |

*Differentiated KG1 cells were treated with the above treatments for 16 hours and then incubated with 40 μg/mL Fluorescein labeled silica beads (50-100 nm [SNP], 30-70 nm [ENP] or 30-70 nm coated with IgG [ENPG]) for 4 hours. At least 4,000 cells for each treatment were analyzed using the Amnis IDEAS software and the distribution of the cell internalization scores were plotted (n = 2). Means with different letters are significantly different from all combinations of pairs by Tukey-Kramer's honest significant difference (HSD; P ≤ 0.05).

Taken together, F_{CBD} induced macrophage activation manifested by secretion of pro-inflammatory cytokines, polarization and phagocytosis. Moreover, a synthetic composition containing phytocannabinoid standards that mimic F_{CBD} had a significantly reduced effect on macrophage activation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Mehta, P. et al. COVID-19: consider cytokine storm syndromes and immunosuppression. Lancet (London, England) 395, 1033 (2020).
2. Shi, Y., et al., COVID-19 infection: the perspectives on immune responses. Cell Death Differ. 27, 1451-1454 (2020).
3. Ruscitti, P., Berardicurti, O., lagnocco, A. & Giacomelli, R. Cytokine storm syndrome in severe COVID-19. Autoimmun. Rev. 19, 102562 (2020).
4. Gao, Y. et al. Diagnostic utility of clinical laboratory data determinations for patients with the severe COVID-19. J. Med. Virol. 92, 791-796 (2020).
5. Hanu, L. O., Meyer, S. M., Muñoz, E., Taglialatela-Scafati, 0. & Appendino, G. Phytocannabinoids: a unified critical inventory. Nat. Prod. Rep. 33, 1357-1392 (2016).
6. Berman, P. et al. A new ESI-LC/MS approach for comprehensive metabolic profiling of phytocannabinoids in Cannabis. Sci. Rep. 8, 1-15 (2018).
7. Aizpurua-Olaizola, O. et al. Evolution of the cannabinoid and terpene content during the growth of Cannabis sativa plants from different chemotypes. J. Nat. Prod. 79, 324-331 (2016).
8. Olah, A., Szekanecz, Z. & Biro, T. Targeting cannabinoid signaling in the immune system: “High”-ly exciting questions, possibilities, and challenges. Front. Immunol. 8, 1487 (2017).
9. Gonçalves, E. D. & Dutra, R. C. Cannabinoid receptors as therapeutic targets for autoimmune diseases: where do we stand? Drug Discov. Today 24, 1845-1853 (2019).
10. Romano, B. et al. Pure Δ9-tetrahydrocannabivarin and a Cannabis sativa extract with high content in Δ9-tetrahydrocannabivarin inhibit nitrite production in murine peritoneal macrophages. Pharmacol. Res. 113, 199-208 (2016).
11. Friedman, M., Cepero, M. L., Klein, T. & Friedman, H. Suppressive effect of Δ9-tetrahydrocannabinol in vitro on phagocytosis by murine macrophages. Proc. Soc. Exp. Biol. Med. 182, 225-228 (1986).
12. Ahmed, A. et al. Ruxolitinib in adult patients with secondary haemophagocytic lymphohistiocytosis: an open-label, single-centre, pilot trial. Lancet Haematol. 6, e630-e637 (2019).
13. Hua, W. et al. CD36 mediated fatty acid-induced podocyte apoptosis via oxidative stress. PloS ONE 10, e0127507 (2015).
14. Gallily, R., Yekhtin, Z. & Hamš, L. O. Overcoming the bell-shaped dose-response of cannabidiol by using cannabis extract enriched in cannabidiol. Pharmacol. Pharm. 6, 75 (2015).
15. Laprairie, R., Bagher, A., Kelly, M. & Denovan-Wright, E. Cannabidiol is a negative allosteric modulator of the cannabinoid CB1 receptor. Br. J. Pharmacol. 172, 4790-4805 (2015).
16. Mukhopadhyay, I. et al. Expression of functional TRPA1 receptor on human lung fibroblast and epithelial cells. J. Recept. Signal Transduct. Res. 31, 350-358 (2011).
17. Merad, M. & Martin, J. C. Pathological inflammation in patients with COVID-19: a key role for monocytes and macrophages. Nat. Rev. Immunol. 20, 355-362 (2020).
18. Remy, K. E. et al. Immunotherapies for COVID-19: lessons learned from sepsis. Lancet Respir. Med. (2020). www(dot)dx(dot)doi(dot)org/10(dot)1016%2FS2213-2600(20)30217-4
19. Levy, Y. et al. Enhanced T cell recovery in HIV-1-infected adults through IL-7 treatment. J. Clin. Invest. 119, 997-1007 (2009).
20. Dalan, R.; et al. The ACE-2 in COVID-19: Foe or friend? Horm. Metab. Res. 52, 257 (2020).
21. Li, F., Li, W., Farzan, M. & Harrison, S. C. Structure of SARS coronavirus spike receptor-binding domain complexed with receptor. Science 309, 1864-1868 (2005).
22. Garvin, M. R. et al. A mechanistic model and therapeutic interventions for COVID-19 involving a RAS-mediated bradykinin storm. elife 9, e59177 (2020).
23. Wang, C. et al. Alveolar macrophage dysfunction and cytokine storm in the pathogenesis of two severe COVID-19 patients. EBioMedicine 57, 102833 (2020).
24. Merad, M. & Martin, J. C. Pathological inflammation in patients with COVID-19: a key role for monocytes and macrophages. Nat. Rev. Immunol. 20, 1-8 (2020).
25. Yoshikawa, T., Hill, T., Peters, C. J. & Tseng, C.-T. K. Severe acute respiratory syndrome-Coronavirus (SARS-CoV)-induced lung epithelial cytokines exacerbate SARS pathogenesis by modulating intrinsic functions of monocyte-derived macrophages and dendritic cells. J. Virol. (2008). www(dot)doi(dot)org/10(dot)1128/JVI(dot)01792-08
26. Yasui, F. et al. Phagocytic cells contribute to the antibody-mediated elimination of pulmonary-infected SARS coronavirus. Virology 454, 157-168 (2014).
27. Horsthemke, M. et al. Multiple roles of filopodial dynamics in particle capture and phagocytosis and phenotypes of Cdc42 and Myo10 deletion. J. Biol. Chem. 292, 7258-7273 (2017).
28. Tsugita, M., Morimoto, N., Tashiro, M., Kinoshita, K. & Nakayama, M. SR-B1 is a silica receptor that mediates canonical inflammasome activation. Cell Rep. 18, 1298-1311 (2017).
29. Greenberg, S. & Grinstein, S. Phagocytosis and innate immunity. Curr. Opin. Immunol. 14, 136-145 (2002).
30. Wang, X., Lv, L., Chen, Y. & Chen, J. A CD36 synthetic peptide inhibits silica-induced lung fibrosis in the mice. Toxicol. Ind. Health 26, 47-53 (2010).
31. He, W. et al. Alveolar macrophages are critical for broadly-reactive antibody-mediated protection against influenza A virus in mice. Nat. Commun. 8, 1-14 (2017).
32. Huber, V. C., Lynch, J. M., Bucher, D. J., Le, J. & Metzger, D. W. Fc receptor-mediated phagocytosis makes a significant contribution to clearance of influenza virus infections. J. Immunol. 166, 7381-7388 (2001).
33. Cooper, G. E. et al. Viral inhibition of bacterial phagocytosis by human macrophages: redundant role of CD36. PLoS One 11, e0163889 (2016).
34. Yip, M. S. et al. Antibody-dependent infection of human macrophages by severe acute respiratory syndrome coronavirus. Virol. J. 11, 1-11 (2014).
35. Merad, M. & Martin, J. C. Pathological inflammation in patients with COVID-19: a key role for monocytes and macrophages. Nat. Rev. Immunol. 20, 355-362(2020).
36. Navarro, S., Debili, N., Bernaudin, J., Vainchenker, W. & Doly, J. Regulation of the expression of IL-6 in human monocytes. J. Immunol. 142, 4339-4345 (1989).
37. Pastor, F. P., Folgar, M. I., Carvalho, N., Carvalho, F. & Horcajadas, F. A. Therapeutic Cannabis and COVID-19: between opportunism and infoxication. Adicciones 32, 167-172 (2020).
38. Mazuz, M., et al. Synergistic cytotoxic activity of cannabinoids from cannabis sativa against cutaneous T-cell lymphoma (CTCL) in-vitro and ex-vivo. Oncotarget 11, 1141 (2020).
39. Starr, T., Bauler, T. J., Malik-Kale, P. & Steele-Mortimer, O. The phorbol 12-myristate-13-acetate differentiation protocol is critical to the interaction of THP-1 macrophages with Salmonella typhimurium. PLoS ONE 13, e0193601 (2018).
40. Nallathambi, R. et al. Anti-inflammatory activity in colon models is derived from δ9-tetrahydrocannabinolic acid that interacts with additional compounds in cannabis extracts. Cannabis Cannabinoid Res. 2, 167-182 (2017).
41. Yaakov, N. et al. Single cell encapsulation via pickering emulsion for biopesticide applications. ACS omega 3, 14294-14301 (2018).
42. Mani, K. A., Yaakov, N., Itzhaik Alkotzer, Y., Zelikman, E. & Mechrez, G. A robust fabrication method for amphiphilic Janus particles via immobilization on polycarbonate microspheres. Polymers 10, 900 (2018).

What is claimed is:

1. A method of treating a disease that can benefit from activating macrophages, wherein the disease is not cancer, in a subject in need thereof, the method comprising administering to the subject a therapeutically effective amount of a composition comprising a cannabis derived fraction comprising phytocannabinoids, wherein said phytocannabinoids comprise at least 80% CBD and at least one of CBG and THCV, wherein when said phytocannabinoids comprise said CBG said phytocannabinoids comprise at least 2% CBG, thereby treating the disease in the subject.

2. The method of claim 1, wherein said disease is selected from the group consisting of infectious disease, systemic lupus erythematosus, rheumatoid arthritis, multiple sclerosis, a lipid metabolism disorder, atherosclerosis, obstructive jaundice, cholestatic liver disease, skin xanthoma, xanthelasma, tuberous xanthoma, xanthoma striatum, vanishing bile duct syndrome.

3. The method of claim 1, wherein said disease is an infectious disease.

4. The method of claim 3, wherein said infectious disease is at a stage prior to cytokine storm.

5. The method of claim 3, wherein said infectious disease is associated with a viral infection.

6. The method of claim 1, wherein said cannabis derived fraction has a pro-inflammatory effect on macrophages.

7. The method of claim 1, wherein said cannabis derived fraction has an anti-inflammatory effect on lung epithelial cells.

8. A method of treating a cytokine storm in a subject in need thereof, the method comprising administering to the subject a therapeutically effective amount of a synthetic composition comprising phytocannabinoids, wherein said phytocannabinoids comprise at least 80% CBD and at least one of CBG and THCV, thereby treating the disease in the subject.

9. The method of claim 8, wherein said cytokine storm is associated with a viral infection.

10. The method of claim 8, wherein said composition has an anti-inflammatory effect on lung epithelial cells.

11. The method of claim 7, wherein said composition has a combined additive or synergistic anti-inflammatory effect on lung epithelial cells as compared to each of said CBD, CBG and THCV when administered as a single agent.

12. The method of claim 10, wherein said composition has a combined additive or synergistic anti-inflammatory effect on lung epithelial cells as compared to each of said CBD, CBG and THCV when administered as a single agent.

13. The method of claim 6, wherein said composition has a combined additive or synergistic pro-inflammatory effect on macrophages as compared to each of said CBD, CBG and THCV when administered as a single agent.

14. The method of claim 5, wherein said viral infection is a respiratory viral infection.

15. The method of claim 14, wherein said viral infection is a Corona virus infection.

16. The method of claim 9, wherein said viral infection is a respiratory viral infection.

17. The method of claim 16, wherein said viral infection is a Corona virus infection.

18. The method of claim 1, wherein said composition is devoid of phytocannabinoids other than said CBD, CBG and/or THCV.

19. The method of claim 8, wherein said composition is devoid of cannabis active ingredients other than said phytocannabinoids.

20. The method of claim 1, wherein said cannabis derived fraction comprises cannabis active ingredients other than said phytocannabinoids.

21. The method of claim 20, wherein said cannabis derived fraction comprises at least one of the terpenes and terpenoids listed in Table 1.


SEQUENCE LISTING (18 sequences)

SEQ ID NO: 1 - DNA, 20 bp, synthetic construct
  misc_feature 1..20: note = Single strand DNA oligonucleotide
  source 1..20: mol_type = other DNA
aagcactcacgattgttggg

SEQ ID NO: 2 - DNA, 20 bp, synthetic construct
  misc_feature 1..20: note = Single strand DNA oligonucleotide
  source 1..20: mol_type = other DNA
caccccaactatctctcgct

SEQ ID NO: 3 - DNA, 20 bp, synthetic construct
  misc_feature 1..20: note = Single strand DNA oligonucleotide
  source 1..20: mol_type = other DNA
aaggagatctgtgctgaccc

SEQ ID NO: 4 - DNA, 20 bp, synthetic construct
  misc_feature 1..20: note = Single strand DNA oligonucleotide
  source 1..20: mol_type = other DNA
gctgcagattcttgggttgt

SEQ ID NO: 5 - DNA, 20 bp, synthetic construct
  misc_feature 1..20: note = Single strand DNA oligonucleotide
  source 1..20: mol_type = other DNA
gaactccttctccacaagcg

SEQ ID NO: 6 - DNA, 20 bp, synthetic construct
  misc_feature 1..20: note = Single strand DNA oligonucleotide
  source 1..20: mol_type = other DNA
gaagaggtgagtggctgtct

SEQ ID NO: 7 - DNA, 20 bp, synthetic construct
  misc_feature 1..20: note = Single strand DNA oligonucleotide
  source 1..20: mol_type = other DNA
caccctccaacatgaaagcc

SEQ ID NO: 8 - DNA, 20 bp, synthetic construct
  misc_feature 1..20: note = Single strand DNA oligonucleotide
  source 1..20: mol_type = other DNA
ggtggtccttctgtagctct

SEQ ID NO: 9 - DNA, 20 bp, synthetic construct
  misc_feature 1..20: note = Single strand DNA oligonucleotide
  source 1..20: mol_type = other DNA
ctgaaagtacactgctggcg

SEQ ID NO: 10 - DNA, 20 bp, synthetic construct
  misc_feature 1..20: note = Single strand DNA oligonucleotide
  source 1..20: mol_type = other DNA
gagttgccgagtctgtgttg

SEQ ID NO: 11 - DNA, 20 bp, synthetic construct
  misc_feature 1..20: note = Single strand DNA oligonucleotide
  source 1..20: mol_type = other DNA
gccaattccactgatcctgt

SEQ ID NO: 12 - DNA, 20 bp, synthetic construct
  misc_feature 1..20: note = Single strand DNA oligonucleotide
  source 1..20: mol_type = other DNA
cctggggttcagagtcatgt

SEQ ID NO: 13 - DNA, 20 bp, synthetic construct
  misc_feature 1..20: note = Single strand DNA oligonucleotide
  source 1..20: mol_type = other DNA
ctgtgggtgagatcatgtgg

SEQ ID NO: 14 - DNA, 20 bp, synthetic construct
  misc_feature 1..20: note = Single strand DNA oligonucleotide
  source 1..20: mol_type = other DNA
gttccacttgtccacgaggt

SEQ ID NO: 15 - DNA, 20 bp, synthetic construct
  misc_feature 1..20: note = Single strand DNA oligonucleotide
  source 1..20: mol_type = other DNA
agatgcagcctcatttccac

SEQ ID NO: 16 - DNA, 20 bp, synthetic construct
  misc_feature 1..20: note = Single strand DNA oligonucleotide
  source 1..20: mol_type = other DNA
tgggttttcaactggagagg

SEQ ID NO: 17 - DNA, 20 bp, synthetic construct
  misc_feature 1..20: note = Single strand DNA oligonucleotide
  source 1..20: mol_type = other DNA
caggaattgaatgggtttgc

SEQ ID NO: 18 - DNA, 20 bp, synthetic construct
  misc_feature 1..20: note = Single strand DNA oligonucleotide
  source 1..20: mol_type = other DNA
aaaccaaggcacagtggaac